(19) United States
(12) Patent Application Publication
(10) Pub. No.: US 2022/0178943 A1
(43) Pub. Date: Jun. 9, 2022

(54) LATERAL FLOW DEVICE

(71) Applicant: FibroTx OÜ, Tallinn (EE)

(72) Inventors: Toomas Neuman, Tallinn (EE); Petrus Johannes Louis Spee, Tallinn (EE); Aram Kazarjan, Tallinn (EE); Ave Laas, Tallinn (EE)

(21) Appl. No.: 17/427,794

(22) PCT Filed: Feb. 6, 2020
(86) PCT No.: PCT/EP2020/052994

§ 371 Date: Aug. 2, 2021

(30) Foreign Application Priority Data
Feb. 6, 2019 (EP) 19155844.4

(51) Int. Cl. G01N 33/68; G01N 33/543; B01L 3/00

(52) U.S. Cl. CPC G01N 33/6869; G01N 33/54388; B01L 3/5023; G01N 2333/545; B01L 2300/105; B01L 2400/0683; G01N 2333/5421; B01L 2300/069

(57) ABSTRACT

The present invention relates to kits and methods based on lateral flow assay devices for detecting the presence or quantity of one or more test analytes within a test sample taken from the skin of a mammal.

### FIELD OF THE INVENTION

The present invention relates to kits and methods based on lateral flow assay devices for detecting the presence or quantity of one or more test analytes within a test sample taken from the skin of a mammal.

### BACKGROUND OF THE INVENTION

Fast development of genomics, transcriptomics, proteomics and regulomics has made it possible to analyze molecular and cellular mechanisms at large scale. One of the important results of these studies has been development of functional genomics and the understanding that cells from different individuals have significant differences in genome structure, gene and protein expression profiles and regulatory mechanisms that control specific cellular functions. This has resulted in an interest in detecting and/or quantifying biomarkers to assess the current state of a mammal by way of presence, absence and/or concentration of one or more biomarkers.

Also there is a need for evaluating how effective treatments are on a personal level, such as in the fields of personalized medicine and personalized skin care.

In relation to personalized skin care the claimed effects of anti-wrinkle and anti-aging effects of cosmetic products are typically based on the assumption that these products have similar effect on all individuals. However, this is not the case. Different people and different skin types react differently to cosmetic products, hence the need for point-of-care devices that can determine the effects or responsiveness of an individual to a particular type of skin care product.

Skin “quality” depends on the biological processes that control and regulate skin morphology, structure and function. Basic biological mechanisms that are responsible for skin performance are related to maintenance, renewal and function of different cell populations in the skin. For example dermal fibroblasts control homeostasis of extracellular matrix, keratinocytes control barrier function of the skin, immune cells and factors are responsible for inflammatory processes and fighting with infections. Functional networks (molecular mechanisms) that control these processes are relatively well known and key players in these networks have been identified. Levels and activity of different cytokines and growth factors regulate balance of cellular processes such as proliferation and differentiation of different cell populations, synthesis and degradation of extracellular matrix, metabolic activity etc. in the skin. Combination of these activities results in the skin “quality” and aesthetic look of the skin.

Levels of interleukins may be used to determine the status of the skin and also provide recommendations how to improve skin “quality” (appearance, function, structure).

One of the challenges faced with lateral flow assay methods are the provision of a sample to test, in particular the provision of a sample form on the skin, and in particular to provide samples from on the skin in a reproducible and/or uniform manner.

WO 2014184151 A1 describes a point-of-care diagnostic device that is based on lateral flow assay technology and enables non-invasive analysis of secreted and diffusible factors from the skin surface.

US 2005/0175992 describes a method for the rapid diagnosis of targets in human body fluids. In particular a lateral flow assay method is employed, where a sample is collected non-invasively from eye fluid using a swab member.

Consequently there is a need in the art for kits and methods for obtaining and analysing analytes from the skin, in particular point-of-care devices that allows for rapid detection.

### SUMMARY OF THE INVENTION

The present invention was made in view of the prior art described above, and the object of the present invention is to provide a kit for detecting the presence or quantity of one or more test analytes within a test sample taken from a skin surface of a mammal.

In a first aspect, the present invention provides a kit for detecting the presence or quantity of one or more test analytes within a test sample obtained from a skin surface of a mammal, the kit comprising:
a) a lateral flow assay device comprising a cassette (300) comprising one or more porous elements forming a porous support assembly (100, 301), wherein said cassette (300) is configured to receive and hold a sample collection pad (201, 101), wherein said sample collection pad (201, 101) is configured to be in contact with said porous support assembly (100, 301) when said sample pad (200, 301) is inserted in said cassette (300),
b) (optionally) a blister pack (302), wherein said blister comprises a buffer solution, wherein said cassette (300) is configured to receive said blister (302), and
c) (optionally) a sample collection pad (201, 101) configured to be used for collecting said test sample.

In second aspect, the present invention provides a method for detecting the presence or quantity of one or more test analytes, the method comprising the following steps:
a) provide a sample collection pad (201, 101) as described herein, such as a separate swab comprising a sample collection pad (201), wherein said sample pad comprises a test sample obtained from the a skin surface of a mammal such as a human being using said separate swab;
b) insert said sample collection pad containing said test sample in the cassette (300) comprising a porous support assembly (100, 301) and blister pack (302) comprising a buffer solution and a housing (306) covering said blister as defined herein,
c) actuating the housing (306) to pierce and dispense said buffer solution, and
d) allowing buffer solution and test sample to migrate through the porous support assembly (100, 301).

### BRIEF DESCRIPTION OF THE DRAWINGS

FIG. 1

FIG. 1 shows perspective views of different embodiments, of the present invention, of a porous support assembly (100) (also referred to as a lateral flow assay strip or a test strip). In FIG. 1a a porous support assembly (100) is shown with a sample collection pad (101), an conjugate pad (102), a detection zone (105) and an indicator zone (106), both zones immobilized on porous support (107), a wicking pad (104) and a backing material (108). “L” shows the direction of the lateral flow and the area “DA” defines the detection area. FIG. 1b illustrates the porous support assembly (100), where the sample collection pad (101) is detached from the remaining porous support assembly. FIG. 1c shows an alternative embodiment of FIG. 1a, where the sample pad (101); conjugate pad (102); detection zone (105) and indicator zone (106) on porous support (107); and wicking pad (104) is adjoining or overlapping, and placed on a backing material (108).

FIG. 2

FIG. 2 shows in an embodiment of the present invention different views of a separate swab (200) comprising a supporting member (202) with an aperture (205) at the distal end (204) of the supporting member (202). The separate swab (200) is disclosed without and with the sample collection pad (201) attached and covering the periphery of the aperture. The supporting member (202) comprises an incision (206) on one of the edges of the supporting member (202). The incision (206) is configured to interact with the bulge in the cassette (300) to orientate and secure the position of the swab in the inserted position in the lateral flow device (300). FIG. 2 further discloses an embodiment of the separate swab (200), where the width of the proximal end (203) of the supporting member (202) is extended to form a finger grip.

FIG. 3

FIG. 3 shows in an embodiment of the present invention the lateral flow device (300) and its parts.

FIG. 4

FIG. 4a shows in an embodiment of the present invention different views of the cassette (300) with the housing in the ready-to-use position (button up position), where the housing (306) covers the blister pack (302). FIG. 4b shows in an embodiment of the present invention different views of the cassette (300) with the housing in the actuated (used) position (button down position), where the housing (306) has been actuated to pierce the blister pack (302) and release the buffer solution.

FIG. 5

FIG. 5 shows in an embodiment of the present invention, the assembled cassette (300) and a separate swab.

FIG. 6

FIGS. 6a and 6b show different views of an embodiment of the present invention, i.e. the lateral flow device (300).

FIG. 7

FIG. 7 shows in an embodiment of the present invention, the blister pack (302)

FIG. 8

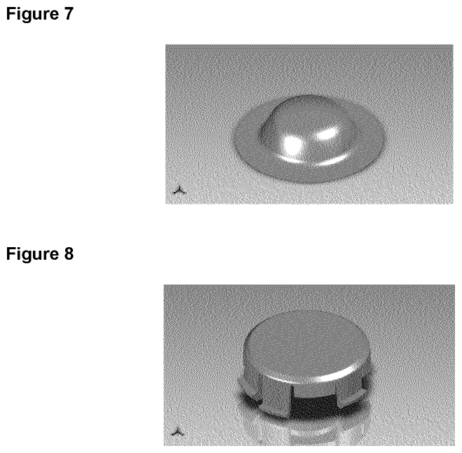

FIG. 7 shows in an embodiment of the present invention, the housing (306) comprising protruding locking members (307, 308).

FIG. 9

Cassettes run with different fill volume blisters as running buffer dispensers. 105 μl blister fill volume—test not run. 125 μl blister fill volume—test started running ˜5 min later, not complete run (background staining). 135 μl blister fill volume—satisfactory test run. 145 μl blister fill volume—satisfactory test run. 155 μl blister fill volume—satisfactory test run. 165 μl blister fill volume—only one of two cassette showed a satisfactory test run.

FIG. 10

Test strips from the cassettes shown in FIG. 9 (run with different fill volume blisters as running buffer dispensers). From the top down: Dry test strip (for comparison only), Run with 105 μl blister, Run with 125 μl blister, Run with 135 μl blister, Run with 145 μl blister, Run with 155 μl blister and Run with 165 μl blister.

FIG. 11

C083 sample collection pad, running buffer released from 145 μl blister. From top down: C083+Standard 1, Correct running; C083+Standard1, Correct running; C083+Standard2, Correct running; C083+Standard, Correct running; C083+Standard3, Running not started, C083+Standard3, Correct running.

FIG. 12

CFP230 sample collection pad with original thin (0.02″) carrier, running buffer released from 145 μl blister. From top down: CFP230 thin+Standard1, Correct running; CFP230 thin+Standard1, Running not started; CFP230 thin+Standard2, Running not started; CFP230 thin+Standard, Correct running; CFP230 thin+Standard3, Running not started; CFP230 thin+Standard3, Running not started.

FIG. 13

CFP230 sample collection pad with thicker (0.04″) carrier, running buffer released from 145 μl blister. From top down: CFP230 0.04″+Standard1, Correct running; CFP230 0.04″+Standard1, Correct running; CFP230 0.04″+Standard2, Correct running; CFP230 0.04″+Standard, Correct running; CFP230 0.04″+Standard3, Correct running; CFP230 0.04″+Standard3, Correct running.

FIG. 14

CFP230 sample collection pad with thicker (0.06″) carrier, running buffer released from 145 μl blister. From top down: CFP230 0.06″+Standard1, Correct running; CFP230 0.06″+Standard2, Correct running; CFP230 0.06″+Standard3, Correct running; CFP230 0.06″+Standard1, Correct running; CFP230 0.06″+Standard2, Correct running; CFP230 0.04″+Standard3, Correct running.

FIG. 15

Skin swabs tested with different sample collection pads, running buffer released from 145 μl blister. From top down: C083+Cheek sample, Correct running; C083+Cheek sample, Late start and incomplete running; CFP230 thin+Cheek sample, Running not started; CFP230 thin+Cheek sample, Running not started; CFP230 0.04″+Cheek sample, Correct running; CFP230 0.06″+Cheek sample, Running not started.

FIG. 16

Test run: 155 μl fill volume blisters and sample collection pads with thin (0.02″) carrier. First column, number three from the top: Failed to run. Second column, number five from the top: Start of run delayed by 10 min.

FIG. 17

Test run: 155 μl fill volume blisters and sample collection pads with a 0.04″ carrier.

FIG. 18

Test run: 155 μl fill volume blisters and sample collection pads with a 0.04″ carrier. First column, first from the top: Failed to run. Second column, third from the top: Failed to run.

FIG. 19

Test run: 165 μl fill volume blisters and sample collection pads with the thin (0.02″) carrier. Second column, third from the top: Failed to run.

FIG. 20

Test run: 165 μl fill volume blisters and sample collection pads with thicker 0.04″ carriers.

FIG. 21

Test run: 165 μl fill volume blisters and sample pads with thicker 0.06″ carriers. First column, last from the top: Start delayed by two minutes.

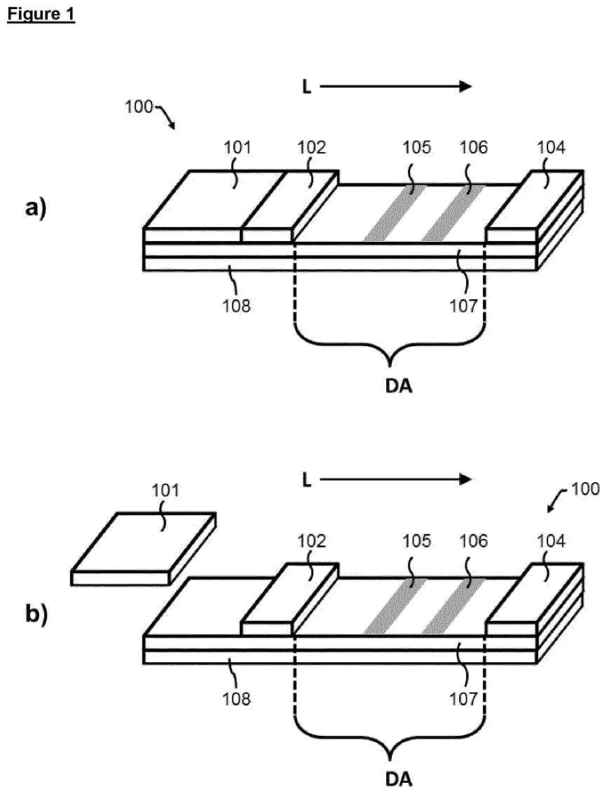

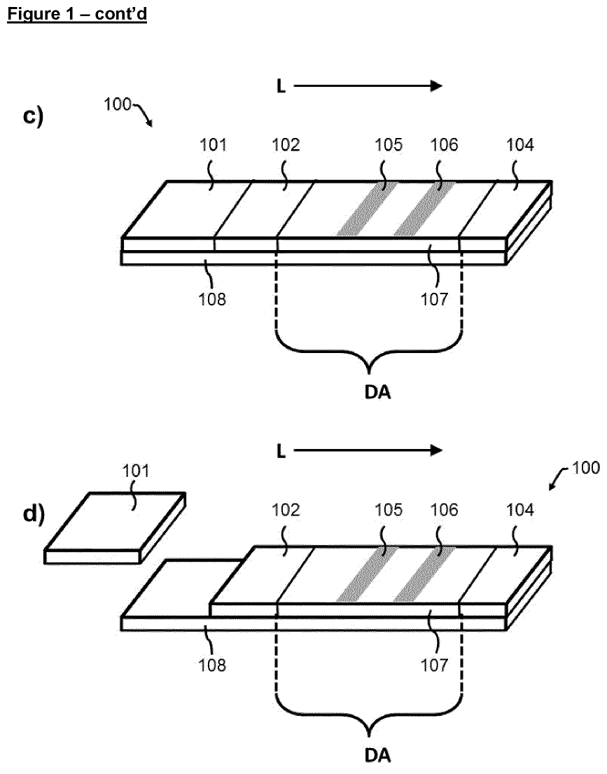

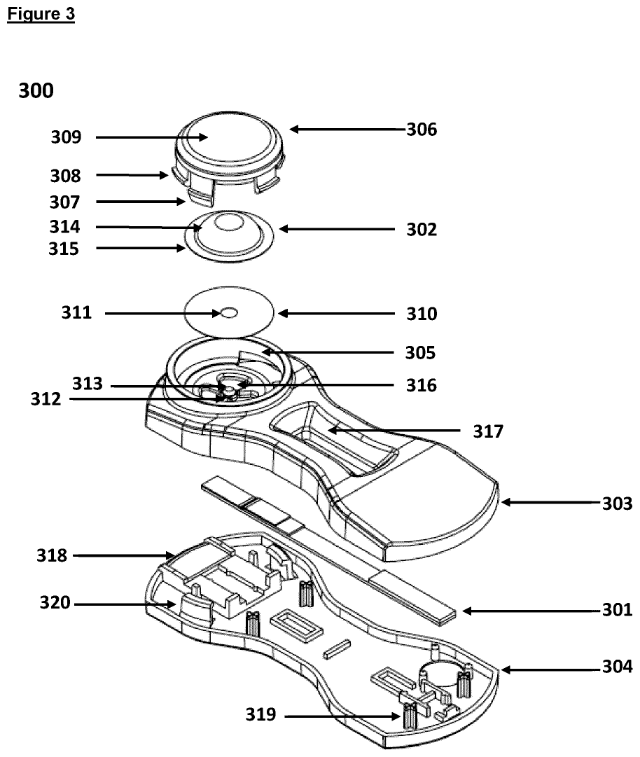

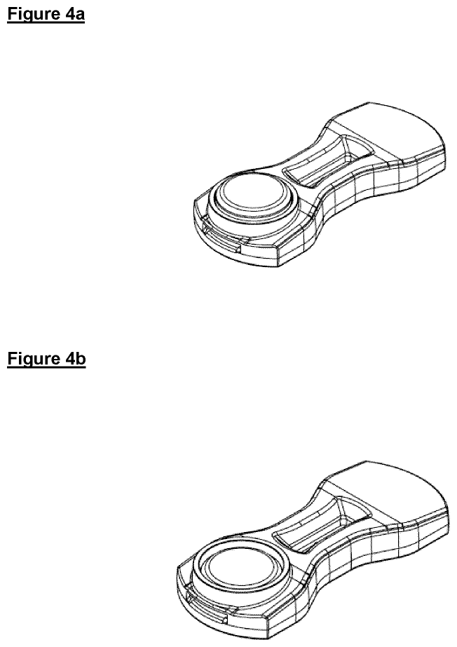

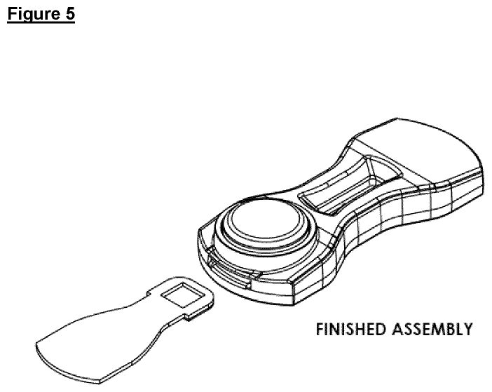

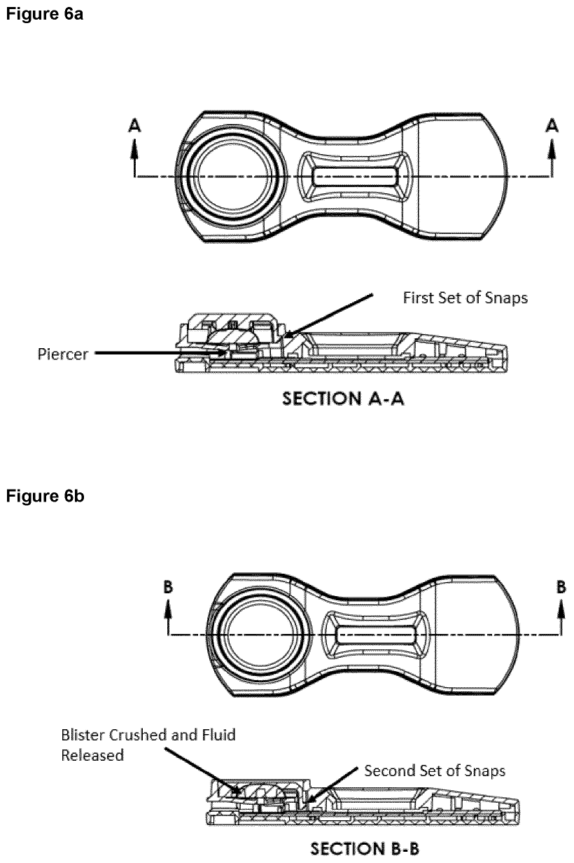

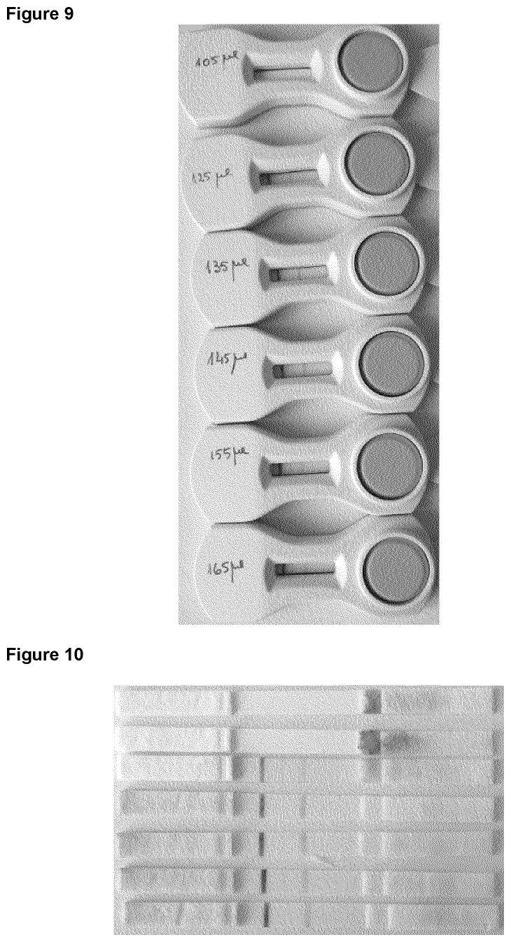

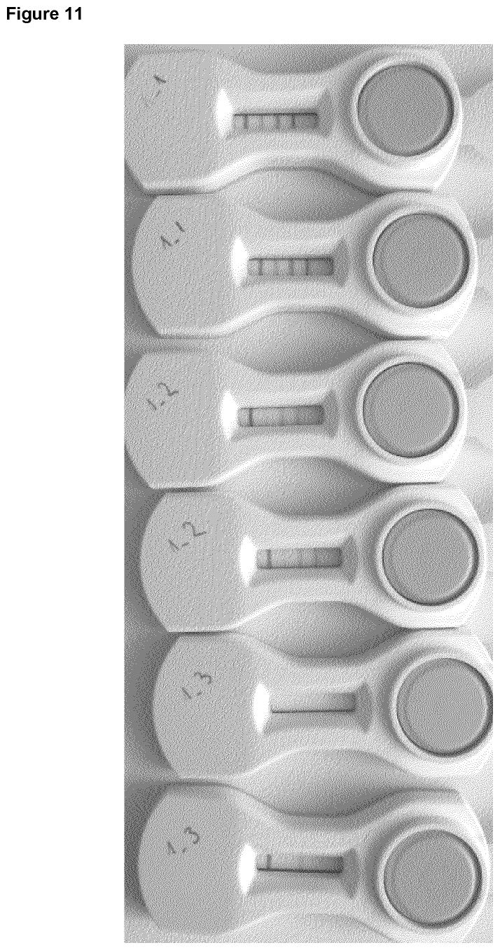

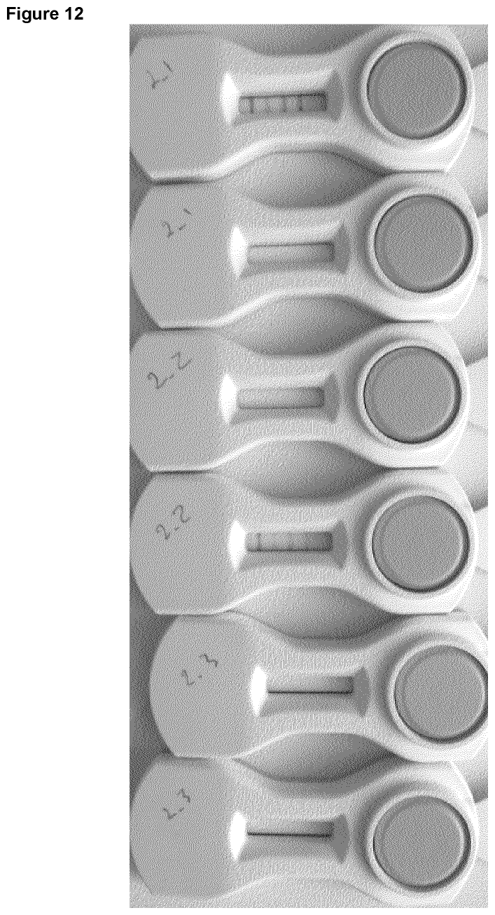

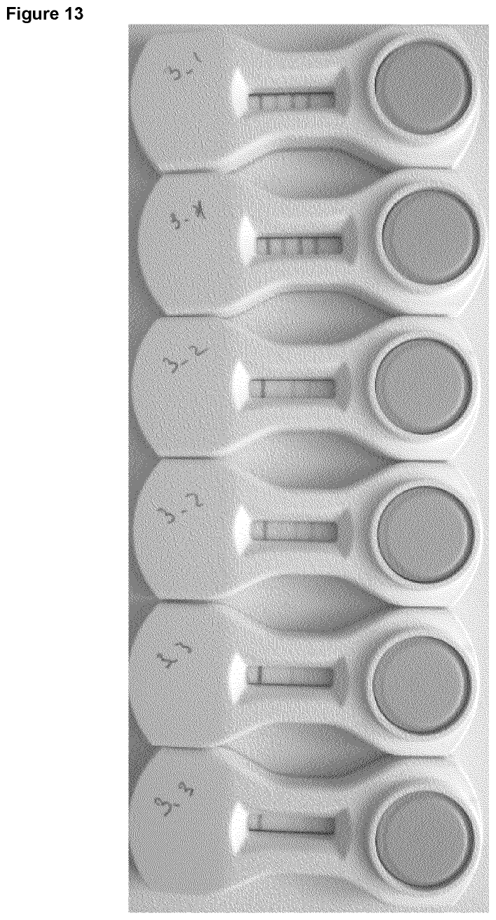

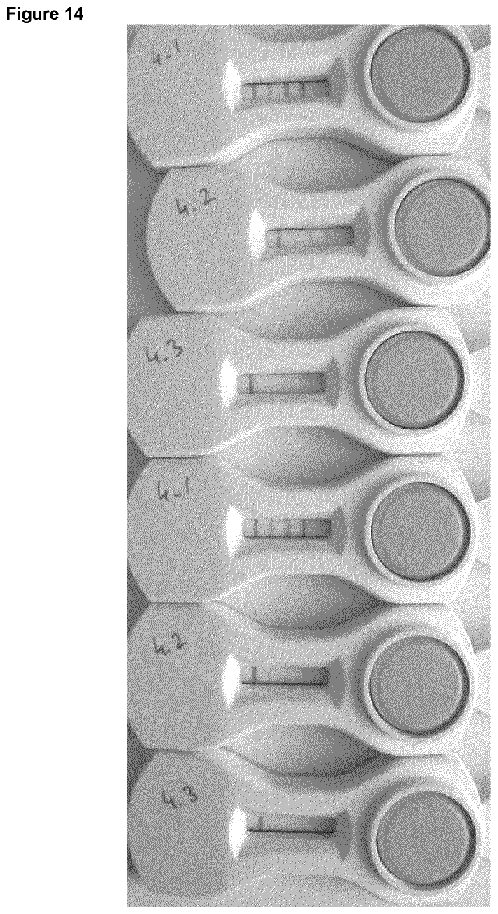

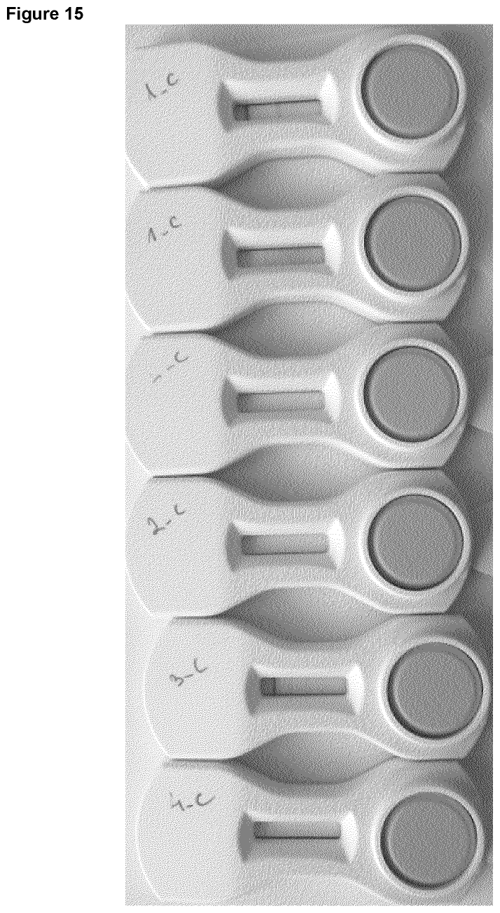

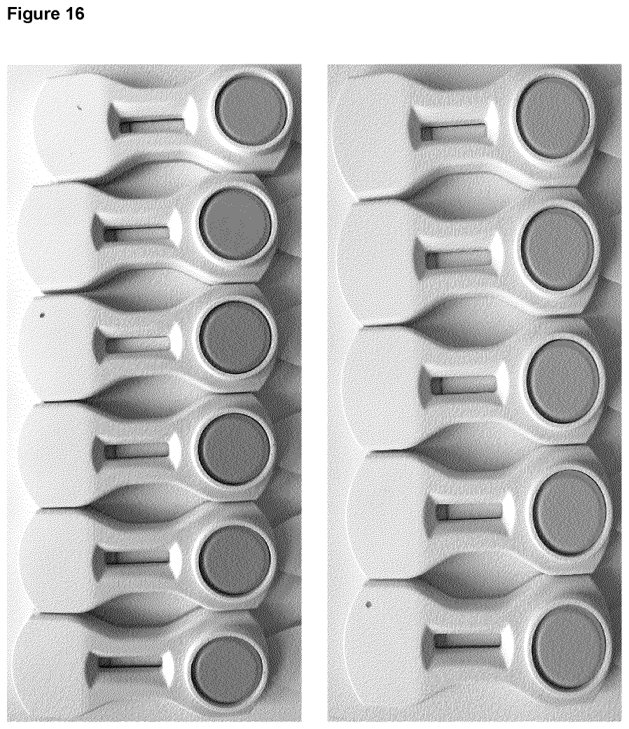

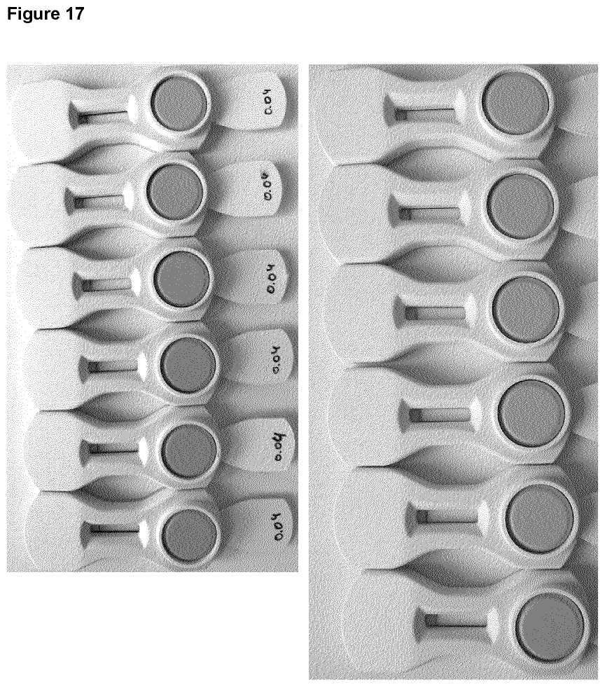

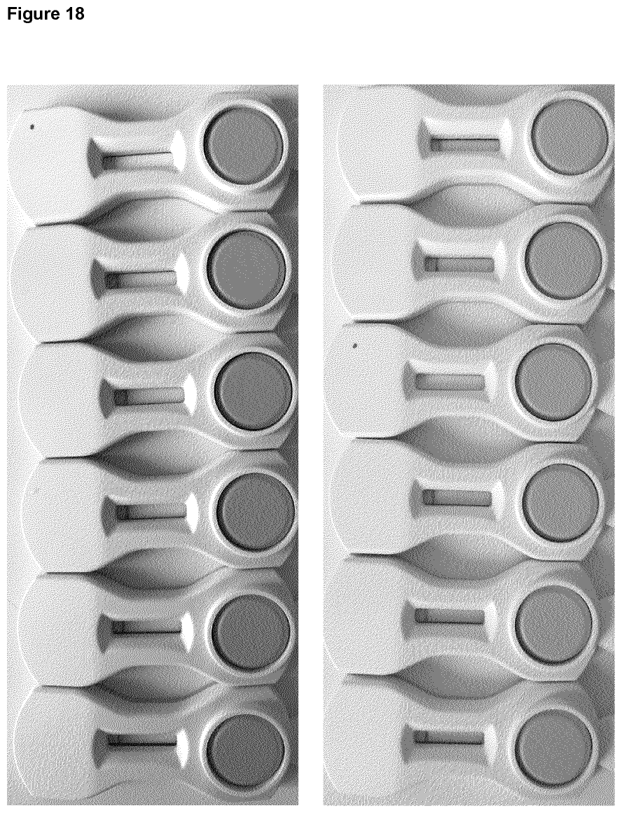

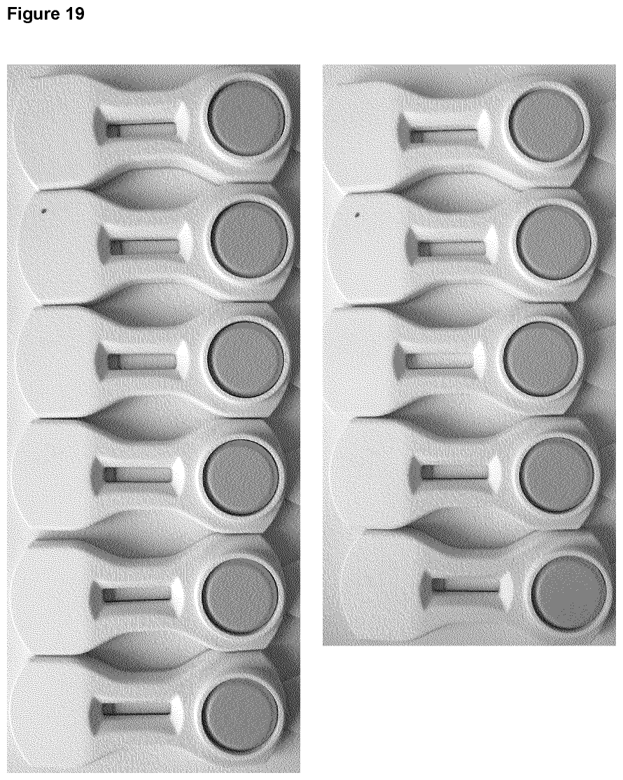

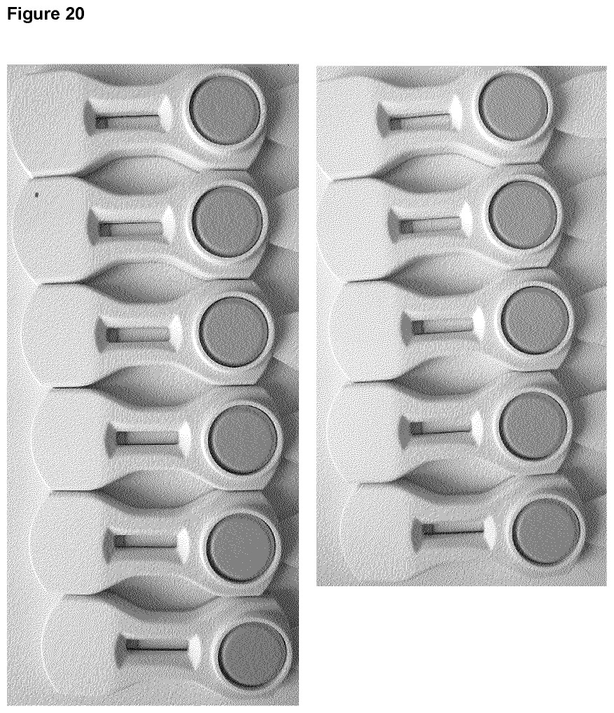

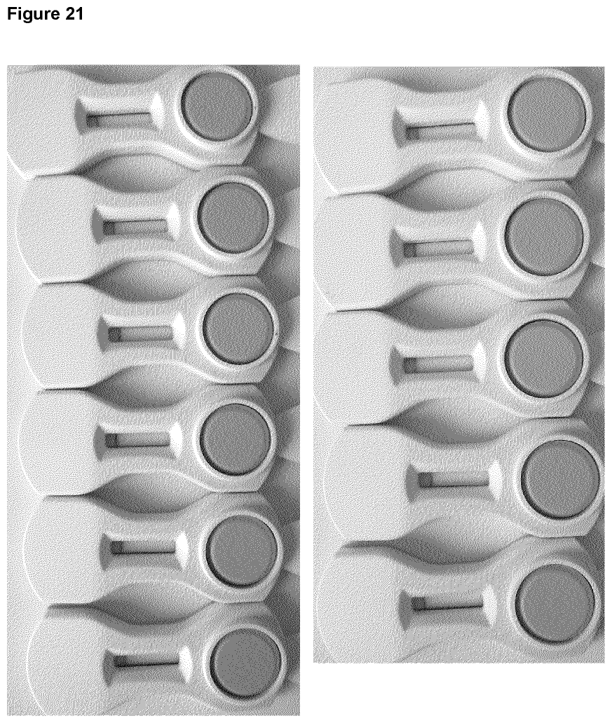

It will be recognized by the person of ordinary skill in the art, given the benefit of this disclosure that certain features shown in FIGS. 1-8 are not necessarily drawn to scale. The dimensions and characteristics of some features in the figures may have been enlarged, distorted or altered relative to other features in the figures to facilitate a better understanding of the illustrative examples disclosed herein.

It will further be recognized by the person of ordinary skill in the art that the individual features of the figures may be interchanged to obtain further embodiments.

### DETAILED DESCRIPTION OF THE INVENTION

In describing the embodiments of the invention specific terminology will be resorted to for the sake of clarity. However, the invention is not intended to be limited to the specific terms so selected, and it is understood that each specific term includes all technical equivalents which operate in a similar manner to accomplish a similar purpose.

Kit for Detecting the Presence of or Quantitating One or More Test Analytes in a Sample

One aspect of the present invention provides a kit for detecting the presence or quantity of one or more test analytes within a test sample obtained from a skin surface of a mammal, the kit comprising:
a) a lateral flow assay device comprising a cassette (300) comprising one or more porous elements forming a porous support assembly (100, 301), wherein said cassette (300) is configured to receive and hold a sample collection pad (201, 101), wherein said sample collection pad (201, 101) is configured to be in contact with said porous support assembly (100, 301) when said sample pad (200, 301) is inserted in said cassette (300),
b) (optionally) a blister pack (302), wherein said blister pack contains a buffer solution, wherein said cassette (300) is configured to receive said blister (302), and
c) (optionally) a sample collection pad (201, 101) configured to be used for collecting said test sample.

In a preferred embodiment, the kit comprises said blister pack (302) and said sample collection pad (201, 101).

It should be understood that the contact with said porous support assembly allows the transfer of a sample from said sample collection pad to said porous support assembly.

In a preferred embodiment, the sample collection pad (201, 101) is provided as a separate swab (200) comprising said sample collection pad (201, 101) as described herein.

The kit of the present invention may be used point-of-care applications to detect the presence or absence of one or more test analytes within a test sample, notably but not restricted to for diagnostic purposes.

The body of the cassette typically comprises or consist of an upper part (303) and a lower part (304). The upper part (303) and the lower part (304) of the assembled cassette is typically hold together by one or more locking members (320). The upper part (303) and/or the lower part (304) may comprise one or more supportive pins (319). The supportive pins (319) contributes to the stability of the assembled cassette (300).

The blister pack typically comprises a blister dome (314) and a blister bottom film (315). The blister pack (302) may be made of a material selected from the group consisting of polypropylene (PP), aluminium, polyethylene terephthalate (PET), polyamide (PA) or a combination thereof. In one embodiment, the blister bottom film (315) is welded or glued to said blister dome (314). In one embodiment, the blister dome (314) has a thickness in the range of 75 to 150 micrometer. In order to ensure that the alignment of the blister pack (302) in the seat (305) is maintained, the blister bottom film (315) may comprise and adhesive that fix the position of the blister pack (302) to the seat (305). Thus in one embodiment, the blister bottom film (315) comprises an adhesive on the outer surface.

The blister comprises a buffer solution that facilitate the migration of the sample through the porous support assembly (100, 301) (also referred to as the test strip). Suitable buffer solutions are well known in the art. Examples of such buffer solutions include but are not limited to PBS (phosphate-buffered saline) buffer, TRIS buffers and casein diluent blocker tween (Senova diluent). The buffer solution may optionally comprise a preservative or biocide. The inventors have discovered that the buffer solution may have an impact on the amount of force needed to actuate the release of the buffer from the blister (302) and the time until the buffer starts migrating through the porous support assembly (100, 301). The physical properties, e.g. viscosity, of the buffer may affect the performance of transferring the buffer from the blister pack onto the porous support assembly (100, 301).

The blister pack (302) shall comprise a volume of buffer solution sufficient to facilitate complete migration of the sample through the porous support assembly (100, 301) and preferably not comprises an excess volume of buffer solution. In one embodiment, the volume of the buffer solution in said blister pack (302) is at least 130 microlitre, such as in the range of 130 to 170 microlitre, preferably in the range of 155 to 165 microlitre.

In a preferred embodiment, the upper part of said cassette comprises a seat (305) configured to receive said blister pack (302). The inventors have discovered that misalignment of the blister in seat may have an adverse effect on the performance of the lateral flow assay. Thus in one embodiment, the diameter of said seat (305) is equal to or approximately equal to but larger than the diameter of the lower part of the blister pack (302), i.e. the diameter of the blister bottom film (315). The seat may be configured such that the wall of the seat are essentially equal to or higher than the distance from the bottom of the blister pack (302) to the highest point of the blister dome (314) such that the blister pack (302) is essentially immersed in the seat (305).

In one embodiment, the cassette (300) comprises a lid configured to cover said blister pack (302) when positioned in the seat (305). In one embodiment, the lid is configured to be pressed against the blister pack (302) to actuate the release of a buffer solution from the blister pack.

In a preferred embodiment, the upper part of said cassette comprises a seat (305) configured to receive said blister pack (302) and a housing (306) covering said blister. In the assembled cassette, the blister pack (302) is positioned in the seat with the housing (306) covering said blister. The kit is preferably provided as an assembled cassette (300) with the blister pack (302) in the seat (302) and the housing (306) covering said blister. Thus in one embodiment, the blister (302) is inserted the seat (305) of the cassette with the housing (306) covering said blister pack. The housing typically comprises and upper part (309) and a lower part. In one embodiment, the housing (306) comprises a least one first (preferably a set of) locking member (307) and a least one (preferably a set of) second locking member (308) protruding from the lower part of the button. In one embodiment, the at least one (preferably a set of) first locking member (307) secures the housing in the cassette in a position covering said blister pack. In this position, the cassette comprising the blister pack (302) is in a ready-to-use state (“button up position”). The first locking member(s) (307) also prevent the cassette from being disassembled by the user. In another embodiment, the at least one (preferably a set of) second locking member (308) secures the housing in the cassette in the actuated position. In this position, the cassette is in an actuated (used) state (“button down position”) comprising the blister pack (302), which has been pierced to release the buffer solution.

The housing is locked in this position and the cassette cannot be mistaken from a cassette, which has not been used. The locking member(s) (308, 307) are typically formed like hooks or the like, which grips into corresponding aperture(s) in seat (305) of the cassette (300). In one embodiment, the at least one first locking member (307) is longer than said at least one second locking member (308).

Thus apart from covering and securing position of the blister pack (302) in the seat (305), the housing (306) or lid is typically configured to be used as a button to actuate the release of buffer solution from the blister (302) by pressing the housing down towards the seat (305).

The inner part of the house may comprises structures which contributes to the alignment of the blister pack (302) in the seat (305). In one embodiment, the housing (306) comprises alignment members configured to align and position the blister pack (302) in the seat (305). In another embodiment, the housing (306) comprises alignment members configured to align and position said blister (302) in the seat (305) and secure an uniform pressure on the blister pack (302).

Apart from covering and securing position of the blister pack (302) in the seat (305), the housing is typically configured to be used as a button to actuate the release of buffer solution from the blister (302) by pressing the housing down towards the seat (305). In one embodiment, the upper part of the housing (309) is configured to prevent the finger from slipping of the button, such as a concave shape, convex shape or rough upper surface of the upper part of the button (309).

The bottom of the seat (305) typically comprises one or more protruding part(s) (312) pointing towards the bottom of the blister pack (302). When the housing is pressed towards the seat (305) one or more protruding part(s) (312) will pierce the blister bottom film (315) and release the buffer solution from the blister (302). Thus in one embodiment, the bottom of the seat comprises at least one protruding part (312). In another embodiment, the protruding part (312) is positioned in the center of said seat (305). In a further embodiment, the protruding part(s) (312) is configured to pierce the bottom part of the blister pack (302) and release the buffer solution from the blister pack (302) when said housing (306) is actuated by pressing the housing towards the seat (305).

In one embodiment, the protruding part(s) (312) comprise a central tubular duct (313) extending from the top of the protruding part to and through the bottom of the seat. The central tubular duct (313) allows the passage of buffer solution from the pierced blister pack (302) to the porous support assembly (301, 100). In another embodiment, the central tubular duct (313) is configured to allow passage of buffer solution from the blister pack (302) when said housing (306) is actuated. The duct may be included to reduce the pressure when the protruding part breaks the blister bottom film (315) and thereby prevent unwanted splashing of the seat (305) and other part of the cassette with buffer solution.

In one embodiment of the present invention, the seat (305) comprises an aperture (316) configured to allow the buffer solution released from the blister pack (302) to come in contact with the porous support assembly (301, 100). The aperture (316) contribute to providing an even dispersion of the buffer solution to the porous support assembly (301, 100) when the buffer is released from the blister pack (302). The seat may comprise one or more apertures (316), such as two, three, four or five apertures (316). The shape of the aperture (316) may for example be round, rectangular, triangular. The seat (305) may for example comprise three aperture (316), such as three aperture (316) having the shape of fan blades, i.e. wide near the periphery of the seat (305) and narrower towards the center.

Preferrably, the cassette comprises a shim (310). In one embodiment, the cassette comprises a seat (305) configured to receive said blister pack (302) and a shim (310), wherein said shim is positioned between the blister pack (302) and the bottom of said seat (305). The shim function as a support for the blister bottom film (315) and contributes to controlling the release of the buffer solution from blister pack (302), such as preventing a burst release of the buffer solution.

In one embodiment, the shim (310) is made of a water impermeable material. In another embodiment, the shim (310) is made of a material selected from the group consisting of polypropylene (PP), aluminium, polyethylene terephthalate (PET), polyamide (PA) or a combination thereof. In another embodiment, the shim (310) is adhesive on the side facing down towards the lower part of the cassette, on the side face up towards the blister or both sides.

In one embodiment, the shim (310) has a central hole (311). The shape and diameter of the hole may vary. In one embodiment, the shim is having a diameter in the range of 2 mm to 5 mm, preferably 3 mm or approximately 3 mm. The size of the hole has an impact on the amount of force needed to actuate the piercing and release of the buffer solution from the blister and the onset of the migration of the buffer/sample in the porous support assembly (301, 100).

In one embodiment, the shim (310) is an integrated member of the seat (305). In another embodiment, the shim (310) is separate part positioned between the blister pack (302) and the bottom of said seat (305).

In one embodiment, the cassette comprises a tablet, capsule or sachet of desiccant. The position of the tablet, capsule or sachet of desiccant may be secured by supporting pins in the bottom part of the cassette (300)

The separate swab of the kit of the present invention is configured to be suitable for collecting a test sample from the skin surface of a mammal. In a preferred embodiment, the mammal is a human being. The swab (200, 301) comprises a supporting member (202) to which a sample collection pad (201, 101) is attached, preferably on one side of the supporting member. The supporting member (202) is typically used as a handle when the sample is collected from the skin, e.g. by placing the sample collection pad (201, 101) on the skin and moving the pad around on the skin using the supporting member (202) to control the movement.

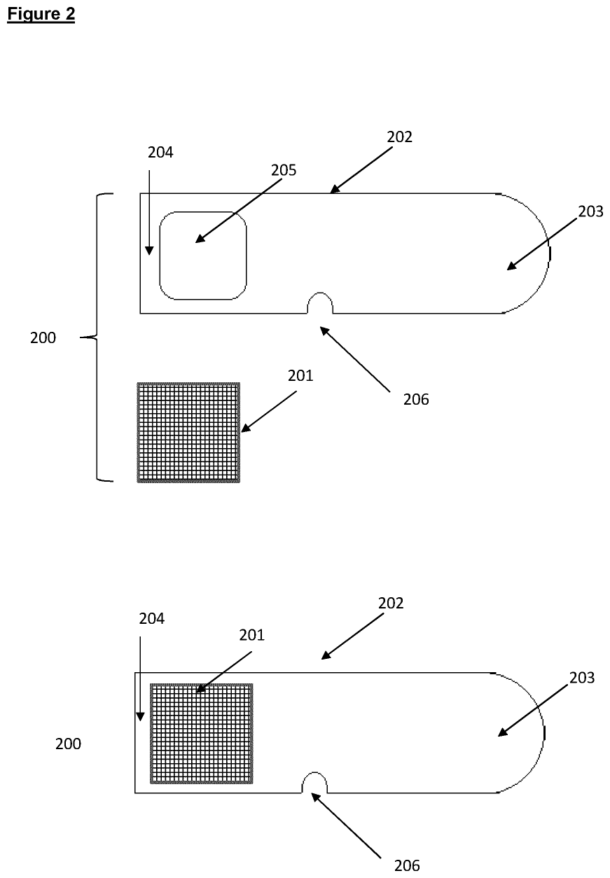

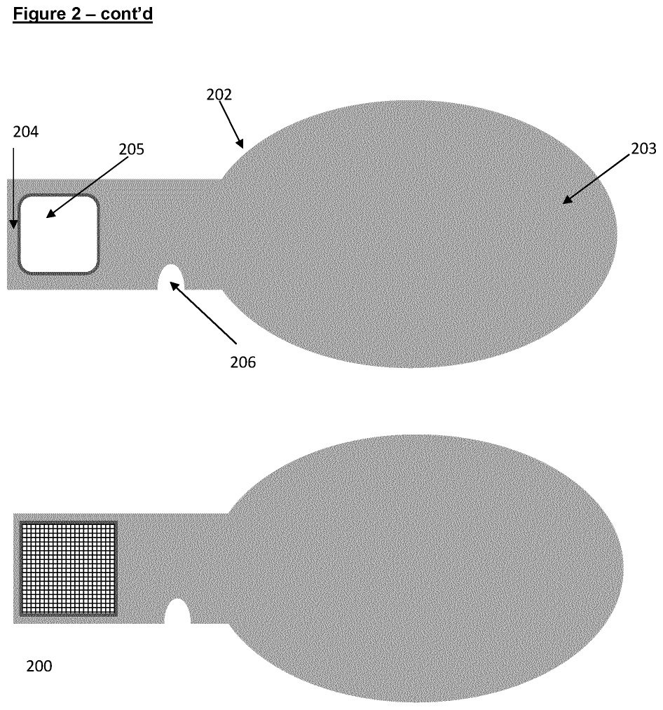

In one embodiment, the supporting member is elongated, for example the length of the member is at least 2 times the width of the member, such as 2.5 times the width of the member, such as 3 times the width of the member, such as at least 4 times the width of the member. In one embodiment, the supporting member is configured with one proximal end (203) configured as a finger grip and opposite distal end (204) to which said sample collection pad (201, 101) is attached. The shape of the proximal end (203) may be configured to allow a firm grip of the supporting member (202) between two or more finger. FIG. 2 discloses an example, where the width of the proximal end (203) of the supporting member (202) is extended to provide a better finger grip. Thus in one embodiment, proximal end (203) of the supporting member is wider than the distal end (204). In one embodiment, the area and shape of the proximal end (203) of the supporting member corresponds to the pulp of a thumb of an adult human being, which allows a firm grip of the supporting member.

In one embodiment, the supporting member (202) is flexible along the longitudinal axis or both axis of supporting member. The supporting member (202) may be made of a material that is flexible material such that the supporting member (202) will bend slightly when the sample collection pad (201, 101) is pressed against the skin and moved around on the skin to collect the sample material. The flexibility of the supporting member (202) reduces the risk of injuring the skin. In a preferred embodiment, the supporting member (202) is made of a plastic material, for example the supporting member (202) may be made of a plastic material, where the thickness of the plastic material is less than about 2 mm, such as 1 mm or less, such as between 2 and 0.5 mm, such as between 2 and 1 mm, which makes the supporting member (202) to flexible along the longitudinal axis.

In a preferred embodiment of the present invention, the distal (204) end of the supporting member (202) comprises an aperture (205) configured to be covered by the sample collection pad (201, 101). In this embodiment, the sample collection pad (201, 101) attached to the supporting member (202) covers aperture and the perimeter of the same. In one embodiment, said sample collection pad (201, 101) is attached to the supporting member (202) such that said sample collection pad covers said aperture (205).

The sample collection pad (201, 101) may be attached to the supporting member (202) close to the perimeter of the aperture. The sample collection pad (201, 101) may be attached to the supporting member (202) further away from the perimeter of the aperture. The sample collection pad (201, 101) is typically attached to one side of the supporting member (202). In one embodiment, the area of the aperture (205) corresponds to at least 50% of the area of the sample collection pad (201, 101), such as at least 60% of the area of the sample collection pad, for example at least 70% of the sample collection pad, such as at least 70% of the sample collection pad, for example at least 80% of the sample collection pad, such as at least 90% of the sample collection pad, for example at least 95% of the sample collection pad.

Inserted in the cassette (300), the sample collection pad (201, 101) of the swab (200) forms part of the porous support assembly (100), i.e. the sample collection pad is in contact with the other elements of the assembly. The aperture allows for access to the sample collection pad (201, 101) for the addition of a running buffer to facilitate the lateral flow in the porous support assembly. In the context of the present invention a running buffer is any liquid buffer suitable for facilitate the lateral flow in the porous support assembly, such as a PBS buffer.

The sample collection pad (201, 101) is made of a material that is suitable for collecting the test sample on the skin and subsequent be mated with and form part of the porous support assembly (100) and release the test sample to the porous support assembly (100). In one embodiment, the sample collection pad (201, 101) is made of a cellulose material, a cellulose derivative such as nitrocellulose, polyether sulfone, polyethylene, nylon polyvinylidene fluoride (PVDF), polyester, polypropylene, glass fibers, cotton, or cloth. In a preferred embodiment, the sample collection pad (201, 101) is made of a cellulose material, a cellulose derivative such as nitrocellulose.

The sample collection pad (201, 101) may be in the form of a sheet or the like. In one embodiment, the sample collection pad (201, 101) is in the form of a layer of one or more sheets or the like, such as a layer of two sheets.

The average thickness the sample collection pad (201, 101) is preferably less than 2 mm, such as in the range of 1 to 0.80 mm, preferably less than 1 mm, such as less than 0.95 mm, for example less than 0.85 mm, such as in the range of 0.85 to 0.80 mm, such as 0.83 mm. In one embodiment, the sample collection pad (201, 101) is in the form of a layer of two sheets, wherein the thickness of each sheet is less than 0.50 mm such as in the range of 0.49 to 0.40 mm.

In order to prevent or reduce any bias between the absorption of the one or more test analytes or any bias in the release of the one or more test analytes from the sample collection pad (201, 101), the sample collection pad (201, 101) may be pre-treated with a blocking buffer (i.e. a buffer that minimize undesired binding of one or more analytes to the sample collection pad). In one embodiment, the blocking buffer is a PBS buffer comprising 1% BSA or a buffer comprising 10 mM Borate, 3% BSA, 1% PVP-40 and 0.25% Triton X100 pH 8.0.

In a particular embodiment, the sample collection pad (201, 101) is in the form of a cellulose material or a cellulose derivative such as nitrocellulose pre-treated with a blocking buffer, wherein the sample collection pad (201, 101) has a thickness in the range of 0.85 to 0.80 mm, such as 0.83 mm.

In embodiment, the cassette (300) comprises a sample port (318) configured to receive said sample collection pad (201, 101).

In one embodiment, the swab (200) is symmetrical. Thus in one embodiment, the insertion of the swab (200) in the sample port (318) of the cassette is not orientation specific. In another embodiment of the present invention, the supporting member (202) of the separate swab (200) comprises an incision (206) on or near the edge of the distal end (204) of said supporting member (202). The cassette (300) comprises a bulge (303) that fits with the incision (206) on the supporting member (202) and orientates and positions the distal end (204) of said supporting member (202) when the swab is inserted in port (318) of cassette (300). Thus, the bulge/incision configuration secures that the swab and in particular the sample collection pad (201, 101) is orientated and positioned correctly in the cassette (300). Thus, the bulge/incision configuration ensures that the swab and in particular the sample collection pad (201, 101) can only be inserted in the cassette (300) such that the the sample collection pad (201, 101) of the swab (200) forms part of the porous support assembly (100), i.e. the sample collection pad is in contact with the other elements of the assembly. Accordingly, in one embodiment, the separate swab (200) comprises an incision (206) on or near the edge of the distal end (204) of said supporting member (202) and the cassette (300) comprises a sample port (318) that comprises a bulge that fits with the incision (206) on the supporting member such that when inserted in the cassette (300), the sample collection pad (201, 101) of the swab (200) forms part of the porous support assembly (100), i.e. the sample collection pad is in contact with the other elements of the assembly.

The sample port (318) may comprise structural elements to seal the port, when the sample collection pad is in the inserted position to prevent backflush of buffer solution through the port (318). In one embodiment, the sample collection pad (201, 101), or sample collection pad (201, 101) attached to the supporting member (202), is having a thickness that prevents buffer solution from leaking out through the sample port (318) when the sample collection pad (201, 101) or sample collection pad (201, 101) attached to the supporting member (202) is inserted in the sample port (318).

In one embodiment, the sample port (318) comprises one or more rails configured position the sample collection pad (201, 101) over and in contact with the said porous support assembly (100, 301). In another embodiment, the cassette (300) comprises a port (318) configured to accept the distal end of said porous support assembly (100, 301) such that the position of the sample collection pad (201, 101, 301) in the cassette (300) is secured.

In the context of the present invention the term lateral flow refers to a liquid flow in which the dissolved or dispersed component(s) of the liquid (including the test analytes) migrates laterally with the liquid through the porous support assembly (100, also referred to as capillary bed, test strip or lateral flow strip) with the proviso that component(s) are not permanently entrapped or by other means excluded from migrating in the liquid. Assay relying on such lateral flow are referred to as lateral flow assay. Where the porous support assembly is preferable made of a non-bibulous material, the components in the liquid will travel at an essential equal speed through the capillary bed. If the porous support assembly is made of a bibulous material, the migration of one of more of the component may be affected by the material. If the porous support assembly comprises or consist of a bibulous material, the material may be treated with a blocking agent, such as PBS buffer comprising BSA, in order to change the properties of the porous support assembly such that the flow characteristics is identical or essentially identical that of a non-bibulous material.

The lateral flow assay is based on the porous support assembly (100)—a capillary bed (such as porous paper or sintered polymer)—having the capacity to transport fluid by action of capillary forces. The porous support assembly (100) is an assembly of porous support elements, which elements are in in fluid communication with each other when fluid (such as a running buffer) is applied to the assembly. One of the porous support elements of the porous support assembly (100) is the sample collection pad (101, 200), which become part of the porous support assembly (100) when the swab is in the inserted position in the cassette (300). The porous support assembly (100) is also referred to as the lateral flow assay strip.

In one embodiment of the present invention, the lateral flow device is constructed so as to form a porous support assembly (100), when it is mated with the sample collection pad attached to said swab, wherein the cassette (300) mated with the sample collection pad comprise an elution zone (101), a conjugate zone (102) and a detection area (DA).

The conjugation zone may be an integrated part of a larger porous element of the porous support assembly (100), such as a porous support strip (107). The conjugation zone may also be in the form of an element of the porous support assembly (100). In a preferred embodiment, the conjugation zone is in the form of a conjugate pad (102).

The sample collection pad (101, 200) functions as a sponge and holds the test sample. Once it is soaked, the test sample, containing one or more test analytes, will migrate from sample collection pad (101, 200) into the adjacent element of the porous support assembly (100). The interphase between the sample collection pad (101, 200) and the adjacent element of the porous support assembly is referred to as the elution zone. The adjacent element of the porous support assembly is typically a conjugate zone, preferably in the form of a conjugate pad (102). The conjugate zone/conjugate pad (102) typically contains one or more indicator affinity molecule(s), such as affinity molecules tagged with detection probe designed to bind to the one or more test analytes within the test sample. The test sample and one or more affinity molecules are mixed and the one or more affinity molecules having affinity for one or more test analytes within the test sample will bind to each other while migrating further to a detection area (DA) that may contain a detection zone (105), and may contain an indicator zone (106), both with one or more stripes, where another set of one or more affinity molecules have been immobilized. By the time the test sample mixed with the affinity molecule(s) from the conjugate pad reaches the detection area (DA), the one or more analytes in the test sample will have been bound to the affinity molecule(s) from the conjugate pad. This complex will then in turn be bound by the affinity molecule(s) on the stripe(s) in the detection zone (105). After a while, when more and more fluid has passed the detection zone, detection probes accumulate, and the stripe changes color. The detection probes may e.g. be gold or latex particles conjugated to the affinity molecule(s) to prepare affinity molecules tagged with detection probes. The detection area (DA) may also comprise an indicator zone (106) which can function as a control to verify that the lateral flow assay has been conducted properly. Such indicator zone (106) may also comprise one or more stripes with affinity molecules immobilized that only binds to the affinity molecule(s) tagged with detection probes from the conjugate pad, whereas the affinity molecule(s) in the detection zone (105) bind to the complex between the analyte(s) and the indicator affinity molecule(s), such as the affinity molecule(s) tagged with detection probes from the conjugate pad. After passing the detection area (DA) the fluid enters the wicking pad (104), which generally receives fluid that has migrated through the entire porous support assembly (100). Thus in one embodiment, the detection area (DA) comprise a detection zone (105) containing one or more affinity molecule(s) for selectively retaining one or more test analyte(s) and optionally an indicator zone (106) containing one or more affinity molecule(s) for selectively retaining one or more indicator affinity molecule(s).

The detection zone (105) may be located upstream or downstream of the indicator zone (106). The lines or stripes in the detector zone or indicator zone may be disposed in a direction that is substantially perpendicular to the flow of the test sample. In some embodiments the lines may be in a direction that is substantially parallel to the flow of the test sample. The lines or stripes in the detection zone (105) or indicator zone (106) does not need to be lines or stripes, and can also be other shapes, such as e.g. dots or patterns.

In one embodiment, the cassette further comprises a wicking pad (104). The wicking pad is part of the porous support assembly (100) and may assist in promoting capillary action and fluid flow from the sample pad (101), conjugate pad (102) through the detection area (DA).

In another embodiment, the cassette comprises a backing material (108) on the backside of said porous support assembly (100) facing away from the elution zone. The backing layer (108) is liquid-impermeable so that fluid flowing through porous support assembly (100) does not leak through the backing layer (108). Examples of suitable materials for the support include, but are not limited to, glass; polymeric materials, such as polystyrene, polypropylene, polyester, polybutadiene, polyvinylchloride, polyamide, polycarbonate, epoxides, methacrylates, and polymelamine.

The porous support assembly (100) is an assembly of two or more porous elements, for example one or more porous elements and the sample collection pad (201, 101), where the swab (200) comprising the sample collection pad is inserted in the lateral flow assay device (300). The elements are preferably in the form of membranes, such as sheet like membranes. The porous support assembly (100) may have a thickness equal to or less than 4 mm (such as less than 4, 3, 2, 1 mm), and a width and a length both greater than the thickness. In some embodiments the width and length of the porous support assembly (100) are both greater (e.g. 3, 4, 5, 6, 7, 8, 9, 10, 50 times greater or up to 4, 5, 6, 7, 8, 9, 10, 50 times greater) than the thickness. In some embodiments the porous support assembly (100) is a square, such as a rectangle, and in some embodiments the porous support assembly (100) is circular. If the porous support assembly (100) is an irregular shape, i.e. different from a square or rectangle, then the width, length and thickness refers to the maximum values for such an irregular shape. For example the width of a circle will be the diameter. Examples of widths and lengths may be 4, 5, 6, 7, 8, 9, 10, 11, 12, 13, 14, 15, 16, 17, 18, 19, 20, 21, 22, 23, 24, 25, 26, 27, 28, 29, 30, 35, 40 mm, such as e.g. range of widths and lengths from 5-30 mm.

Thus in one embodiment, the porous support assembly (100) of the cassette (300) has an average thickness equal to 4 mm or less, and a width and a length, both greater than the thickness, wherein cassette is configured to have a lateral flow direction (L) in the direction of a plane created by the width and the length of the porous support assembly.

In embodiment, the porous support assembly (100, 301) comprises a detection area (DA) and said cassette (300) comprises an inspection window (317). In a further embodiment, the inspection window (317) configured for visual inspection of the detection area (DA). In another embodiment, the inspection window (317) is configured to secure incoming light on whole area of the detection area (DA). In yet another embodiment, the upper surface of the upper part (303) of the cassette (300) comprises at least one reference point suitable for image detection of the detection area (DA) and/or image detection of the orientation of the detection area (DA), such as printed marks and/or shape of the inspection window (317) or shape of the cassette (300).

Method for detecting the presence of or quantitating one or more test analytes in a sample.

One aspect of the present invention relates to a method for detecting the presence or quantity of one or more test analytes, the method comprising the following steps:
a) provide a sample collection pad (201, 101) as defined herein, wherein said sample collection pad comprises a test sample obtained from the a skin surface of a mammal such as a human being using said sample pad;
b) insert said sample collection pad (201, 101) containing said test sample in the cassette (300) comprising a porous support assembly (100, 301) and blister pack (302) comprising a buffer solution and a housing (306) covering said blister as defined herein,
c) actuating the housing (306) to pierce and dispense said buffer solution, and
d) allowing buffer solution and test sample to migrate through the porous support assembly (100, 301).

The assay is allowed develop before the result is evaluated, i.e. it is determined whether the one or more test analytes are present in the sample and optionally the quantity of the one or more test analytes is determined.

In one embodiment, said sample collection pad (201, 101) is attached to a supporting member (202) to provide a separate swab (200).

The subject is a mammal, preferably a human being. The test sample may be obtained using a sample collection pad (201), such as a separate swab (200) comprising a sample collection pad (201), which is applied to the skin of the mammal, preferably the skin of a human being. The area of the skin may for example be the forehead, cheek, the inner arm or a part of the arm which is normally exposed to the sun. The separate swab may be applied to a pre-determined area, such an area not exceeding 5 cm2. The separate swab may also be applied in pre-determined time, such 5 seconds or 30 seconds. The test sample may also be collected by applying a pre-determined motion of the swab, such as a z-shaped motion of the swab on the skin.

In order to prevent or reduce any bias between the absorption of the one or more test analytes or any bias in the release of the one or more test analytes from the sample collection pad (201, 101), the sample collection pad (201, 101) may be pre-treated with a blocking buffer. In one embodiment, the blocking buffer is a PBS buffer comprising 1% BSA or a buffer comprising 10 mM Borate, 3% BSA, 1% PVP-40 and 0.25% Triton X100 pH 8.0.

The sampling may be assisted by wetting the sample collection pad (201) with a fixed volume of fluid. In a preferred embodiment, the sample collection pad (201) of the separate swab (200) is pre-wetted with a buffer before the sample collection, such as a with a fixed volume of a buffer. The buffer may be any suitable buffer such as a PBS buffer. The buffer used for pre-wetting the sample collection pad may be the same buffer used as running buffer in the lateral flow assay step of the procedure.

In another preferred embodiment, running buffer is added to sample collection pad inserted in the cassette (300). The running buffer is added to the sample collection pad inserted in the lateral flow assay device to facilitate or provide sufficient fluid for the lateral flow in the porous support assembly (100) and the development of the assay.

The kit of the present invention may be used for testing analytes present on the skin that have been obtained using a sample collection pad (201, 101), such as a separate swab (200), where the sample collection pad (201, 101) is attached to a supporting member (202). In one embodiment, the one or more test analyte(s) are selected from the list consisting of: chemokines, interleukins, growth factors, hormones, enzymes, and other molecules present on the skin of a mammal, such as selected from the list consisting of: IL-1a, IL-1b, IL-1 RA, IL-8, CCL-2, CCL-5, CCL-27, CXCL-1, CXCL-2, CXCL-9, Trappin2/Elafin, hBD-1, hBD-2, VEGF, and TSLP. In a preferred embodiment the test analytes are the combination of IL-8, IL-1a and IL-1 RA.

As mentioned, the kit of the present invention may for point-of-care application to detect the presence or absence of one or more test analytes within a test sample obtained from the skin using a sample collection pad (201, 101) as defined herein. The readout may be done visually, i.e. presence or absence of a one or more coloured test lines also referred to as test stripes in a detection zone (105), and the confirmation/validation of the test may be done by the presence and/or absence of one or more coloured indicator lines/stripes in an indicator zone (106). The test may be qualitative (presence or absence) as well as quantitative, and the detection/quantification may be aided by reading equipment, or can be purely visual detection by the eye of the user of the lateral flow assay.

If desired, a suitable reading equipment, such as an optical reader may be used in some embodiments to measure the intensity of the probes. The actual configuration and structure of the optical reader may generally vary depending on the probes, which are to be measured. For example, optical detection techniques that may be utilized include, but are not limited to, luminescence (e.g. fluorescence, phosphorescence, etc.), absorbance (e.g. fluorescent or non-fluorescent), diffraction, and so on. Qualitative, quantitative, or semi-quantitative determination of the presence or concentration of an analyte may be achieved in accordance with the present invention. For instance, the amount of the analyte may be quantitatively or semi-quantitatively determined by using the intensities of the signals produced by detection probes bound at the detection zone (105) and the indicator zone (106).

In a preferred embodiment, an image of the detection area (DA) is captured using a suitable device for capturing images, such as a cell-phone comprising a camera. The image may subsequently be transmitted to a computer system (for example a remotely located server) comprising an image processor and a database, where the image is analysed, e.g. by extracting the image features and compare the features with corresponding features stored in a database. The computer system may then generate an output datum based on said image features, which may be transmitted to the user, e.g. back to the cell-phone used for capturing image.

Thus in one embodiment of the present invention, the method of the invention further comprises a step e) of capturing an image of the detection area (DA) and transmitting said image to a computer system comprising an image processor and a database, wherein the image features are extracted from the image by the image processor and said image features is stored in said database and, wherein said computer system generates a least one output datum based on said image features. In a further embodiment, the image is captured using a mobile device, such a cell phone configured to capture images. In yet a further embodiment, the output datum generated by the computer system is transferred to the mobile device.

In yet another embodiment, the upper surface of the upper part (303) of the cassette (300) comprises at least one reference point suitable for image detection of the detection area (DA) and/or image detection of the orientation of the detection area (DA).

Thus in one embodiment of the present invention, the method of the invention further comprises a step e) of capturing an image of the detection area (DA) and transmitting said image to a computer system comprising an image processor and a database, wherein the upper surface of the upper part (303) of the cassette (300) comprises at least one reference point suitable for image detection of the detection area (DA) and/or image detection of the orientation of the detection area (DA), which is recognised by the device for capturing said image. In one embodiment, said at least one reference point is printed marks and/or shape of the inspection window (317) or shape of the cassette (300).

A further aspect of the present invention relates to a method for evaluating a skin condition of a subject at an electronic device comprising a camera, said method comprising the steps of:
obtaining via a user interface, a request for evaluation of the skin condition of a subject,
obtaining via the user interface, user input comprising:
data identifying said subject,
at least one image of the detection area (DA) of a test device used for detecting one or more test analytes, preferably the detection area (DA) of the lateral flow device of the present invention used for detecting one or more test analytes, obtained from the surface of the skin of a mammal, preferably a human being, said at least one image being recorded using the camera arranged at a distance from the test device;
registering said subject based on the data identifying the subject,
processing the user input including said at least one image and
generating an evaluation of the skin condition of a subject;

outputting said evaluation of the skin condition and/or outputting a recommendation for skin care corresponding to the skin condition to the user interface.

In one embodiment, the method further comprises obtaining via the user interface, user input comprising
at least one objective input data parameter on said subject selected from the list consisting of gender and age;
processing the user input including said at least one objective input data parameter.

In a further embodiment, the method further comprises obtaining via the user interface, user input comprising
at least one subjective input data parameter on said subject selected from the list consisting of skin dryness and skin care routines;
processing the user input including said at least one subjective input data parameter.

In one embodiment, the method further comprises obtaining via the user interface, user input comprising
at least one input data parameter on the habitat of said subject selected from the list consisting of temperature, humidity, sun hours, and pollution;
processing the user input including said at least one subjective input data parameter on the habitat of said subject.

In a further embodiment, said evaluation of the skin condition outputted to the user interface includes a recommendation for skin care. In one embodiment, said camera is configured to detect a predetermined shape of detection area of the test device. In a further embodiment, said camera is configured to detect the shape of the inspection window configured for visual inspection of a detection area on said immunochromatographic test strip of a lateral flow device.

In one embodiment, said detection area comprises one or more detection zone(s) containing one or more affinity molecule(s) for selectively retaining one or more test analyte(s) and generating a visible marker such as a spot or a band for each test analyte present.

In another embodiment, the processor of the computing device is configured for detecting the intensity of said marker and assignment of a numerical value for each detected marker based on the marker intensity.

In a further embodiment, said evaluation of the skin condition to the user interface is displayed graphically. In one embodiment, the user input is stored in a database. In another embodiment, the processor is configured to output a predefined evaluation text or graphical presentations corresponding to evaluation of the skin condition. A yet another embodiment, the processor is configured to output a predefined recommendation for skin care corresponding to the skin condition. In a further embodiment, The processor is configured to output a predefined recommendation for skin care corresponding to the skin condition, where said predefined recommendation is retrieved from a database comprising a plurality of recommendations for said skin condition proposing a skin care regimen, wherein proposed skin care regimen is identical and each recommendation of said plurality of recommendations represent alternative wordings of the recommendation.

In one embodiment, the processor is configured to include a training recommendation network, retrieving a skin care regimen from a database comprising a predefined skin care regimen for a predefined skin condition, using said training recommendation network to generate a recommendation for skin care including said skin care regimen outputting said recommendation for skin care to the user interface.

In another embodiment, the training recommendation network uses at least one input parameter selected from the group consisting of said objective input parameter, said subjective input parameter and said input parameter on the habitat of said subject.

In another embodiment, the electronic device is interconnected with a server computer for processing said user input and generating the evaluation of the skin condition. In a further embodiment, the electronic device is interconnected with a server computer for processing said user input and generating the evaluation of the skin condition, wherein said server computer is selected from the group consisting of a cloud and remote server computer.

In one embodiment, the electronic device comprising a camera is a handheld/portable device (such as smartphone). In a further embodiment, the electronic device is a handheld/portable device with a operation system selected from iOS or Android.

A further aspect relates to a system comprising a server computer configured to perform the method of any of the preceding claims.

Yet a further aspect relates to a computer-readable medium encoded with a program to perform the method of any of the preceding claims when run on a computer system.

The terms “comprising”, “comprise” and “comprises” herein are intended by the inventors to be optionally substitutable with the terms “consisting of”, “consist of” and “consists of”, respectively, in every instance.

In describing the embodiments of the invention specific terminology will be resorted to for the sake of clarity. However, the invention is not intended to be limited to the specific terms so selected, and it is understood that each specific term includes all technical equivalents which operate in a similar manner to accomplish a similar purpose.

The invention is further described in the following non-limiting items:

Item 1. A kit for detecting the presence or quantity of one or more test analytes within a test sample obtained from a skin surface of a mammal, the kit comprising:
a) a lateral flow assay device comprising a cassette (300) comprising one or more porous elements forming a porous support assembly (100, 301), wherein said cassette (300) is configured to receive and hold a sample collection pad (201, 101), wherein said sample collection pad (201, 101) is configured to be in contact with said porous support assembly (100, 301) when said sample pad (200, 301) is inserted in said cassette (300),
b) a blister pack (302), wherein said blister pack contains a buffer solution, wherein said cassette (300) is configured to receive said blister pack (302), and
c) a sample collection pad (201, 101) configured to be used for collecting said test sample.

Item 2. The kit according to item 1 characterized in that said cassette comprises an upper part (303) and a lower part (304).

Item 3. The kit according to item 1 or 2 characterized in that the upper part of said cassette comprises a seat (305) configured to receive said blister pack (302).

Item 4. The kit according to any one of the preceding items characterized in that the upper part of said cassette comprises a seat (305) configured to receive said blister pack (302) and a housing (306) covering said blister.

Item 5. The kit according any one of the preceding items characterized in that the diameter of said seat (305) is equal to or approximately equal to the diameter of the lower part of the blister pack (302).

Item 6. The kit according to any one of the preceding items characterized in that the housing comprises an upper part (309) and a lower part.

Item 7. The kit according to any one of the preceding items characterized in that the housing (306) comprises a least one first locking member (307) and a least one second locking member (308) protruding from the lower part of the housing.

Item 8. The kit according to any one of the preceding items characterized in that said at least one first locking member (307) secures the housing in the cassette in a position covering said blister pack (“button up position”).

Item 9. The kit according to any one of the preceding items characterized in that said at least one second locking member (308) secures the housing in the cassette in the actuated position (“button down position”).

Item 10. The kit according to any one of the preceding items characterized in that said at least one first locking member (307) is longer than said at least one second locking member (308).

Item 11. The kit according to any one of the preceding items characterized in that said housing (306) comprises alignment members configured to align and position the blister pack (302) in the seat (305).

Item 12. The kit according to any one of the preceding items characterized in that said housing (306) comprises alignment members configured to align and position said blister (302) in the seat (305) and secure an uniform pressure on the blister pack.

Item 13. The kit according to any one of the preceding items characterized in that said blister pack (302) is inserted into the seat (305) of the cassette with the housing (306) covering said blister pack.

Item 14. The kit according to any one of the preceding items characterized in that said housing (306) comprises a upper part of the housing (309) configured to prevent the finger from slipping of the button, such as a concave shape, convex shape or rough upper surface of the upper part of the button.

Item 15. The kit according to any one of the preceding items characterized in that the upper part (303) of said cassette comprises a seat (305) configured to receive said blister pack (302) and a shim (310), wherein said shim is positioned between the blister pack (302) and the bottom of said seat (305).

Item 16. The kit according to any one of the preceding items characterized in that said shim (310) is made of a water impermeable material.

Item 17. The kit according to any one of the preceding items characterized in that said shim (310) is made of a material selected from the group consisting of polypropylene (PP), aluminium, polyethylene terephthalate (PET), polyamide (PA) or a combination thereof.

Item 18. The kit according to any one of the preceding items characterized in that said shim (310) is adhesive on the side facing down towards the lower part of the cassette, on the side face up towards the blister or both sides.

Item 19. The kit according to any one of the preceding items characterized in that said shim (310) has a central hole (311).

Item 20. The kit according to any one of the preceding items characterized in that said central hole (311) of the shim is having a diameter in the range of 2 mm to 5 mm, preferably 3 mm.

Item 21. The kit according to any one of the preceding items characterized in that said shim (310) is an integrated part of the seat (305).

Item 22. The kit according to any one of the preceding items characterized in that the bottom of the seat comprises at least one protruding part (312).

Item 23. The kit according to any one of the preceding items characterized in that the bottom of the seat comprises a protruding part (312) positioned in the center of said seat (305).

Item 24. The kit according to any one of the preceding items characterized in that said protruding part(s) (312) comprises a central tubular duct (313) extending from the top of the protruding part to and through the bottom of the seat.

Item 25. The kit according to any one of the preceding items characterized in that said protruding part(s) (312) is configured to pierce the bottom part of the blister pack (302) and release the buffer solution from the blister pack (302) when said housing (306) is actuated by pressing the housing towards the seat (305).

Item 26. The kit according to any one of the preceding items characterized in that said protruding part(s) (312) comprising a central tubular duct (313) is configured to allow passage of buffer solution from the blister pack (302) when said housing (306) is actuated.

Item 27. The kit according to any one of the preceding items characterized in that comprises a lid configured to cover said blister pack (302) when positioned in the seat (305).

Item 28. The kit according to any one of the preceding items characterized in that said blister pack (302) comprises blister dome (314) and a blister bottom film (315).

Item 29. The kit according to any one of the preceding items characterized in that the blister pack (302) is made of a material selected from the group consisting of polypropylene (PP), aluminium, polyethylene terephthalate (PET), polyamide (PA) or a combination thereof.

Item 30. The kit according to any one of the preceding items characterized in that said blister dome (314) has a thickness in the range of 75 to 150 micrometer.

Item 31. The kit according to any one of the preceding items characterized in that said blister bottom film (315) is welded to said blister dome (314).

Item 32. The kit according to any one of the preceding items characterized in that said blister bottom film (315) comprises an adhesive on the outer surface.

Item 33. The kit according to any one of the preceding items characterized in that said blister comprises a buffer solution selected from the group consisting of PBS and casein diluent blocker tween, preferably PBS, wherein said buffer solution optionally comprises a preservative or biocide.

Item 34. The kit according to any one of the preceding items characterized in that the volume of the buffer solution in said blister pack (302) is at least 130 microlitre, such as in the range of 130 to 170 microlitre, preferably in the range of 155 to 165 microlitre.

Item 35. The kit according to any one of the preceding items characterized in that said seat (305) comprises an aperture (316) configured to allow the buffer solution released from the blister pack (302) to come in contact with the porous support assembly (301, 100).

Item 36. The kit according to any one of the preceding items characterized in that said aperture (316) is configured to provide an even dispersion of the buffer solution to the porous support assembly (301, 100) when the buffer is released from the blister pack (302).

Item 37. The kit according to any one of the preceding claims, characterized in that said porous support assembly (100, 301) comprises a detection area (DA) and said cassette (300) comprises an inspection window (317) configured for visual inspection of the detection area (DA).

Item 38. The kit according to item 37 characterized in that said inspection window (317) is configured to secure incoming light on whole area of the detection area (DA).

Item 39. The kit according to any one of the preceding items characterized in that said upper surface of the upper part (303) of the cassette (300) comprises at least one reference point suitable for image detection of the detection area (DA) and/or image detection of the orientation of the detection area (DA), such as printed marks and/or shape of the inspection window (317) or shape of the cassette (300).

Item 40. The kit according to any one of the preceding items characterized in that cassette comprises a tablet, capsule or sachet of desiccant.

Item 41. The kit according to any one of the preceding items characterized in that said cassette comprises a sample port (318) configured to receive said sample collection pad (201, 101).

Item 42. The kit according to any one of the preceding items characterized in that said sample collection pad (201, 101) is attached to a supporting member (202).

Item 43. The kit according to any one of the preceding items characterized in that said sample collection pad (201, 101), or sample collection pad (201, 101) attached to the supporting member (202), is having a thickness that prevents buffer solution from leaking out through the sample port (318) when the sample collection pad (201, 101) or sample collection pad (201, 101) attached to the supporting member (202) is inserted in the sample port (318).

Item 44. The kit according to any one of the preceding items characterized in that said sample port (318) comprises one or more rails configured position the sample collection pad (201, 101) over and in contact with the said porous support assembly (100, 301).

Item 45. The kit according to any one of the preceding items, characterized in that one edge of the said distal end (204) of said supporting member (202) comprises an incision (206) and wherein the port (318) of the cassette comprises a bulge configured to orientate and position the distal end of said supporting member when the swab is inserted in the cassette.

Item 46. The kit according to any one of the preceding items, characterized in that the separate swab (200) comprises an incision (206) on or near the edge of the distal end (204) of said supporting member (202) and port (318) of the cassette (300) comprises a bulge that fits with the incision (206) on the supporting member such that when inserted in the cassette (300), the sample collection pad (201, 101) of the swab (200) forms part of the porous support assembly (100).

Item 47. The kit according to any one of the preceding items, characterized in that said distal (204) end of said supporting member (202) comprises an aperture (205), wherein said sample collection pad (201, 101) is attached to the supporting member (202) such that said sample collection pad covers said aperture (205).

Item 48. The kit according to any one of the preceding items, characterized in that said is supporting member (202) is flexible along the longitudinal axis of supporting member.

Item 49. The kit according to any one of the preceding items, characterized in that the supporting member (202) is be made of a material that is flexible material such that the supporting member (202) will bend slightly when the sample collection pad (201, 101) is pressed against the skin and moved around on the skin to collect the test sample.

Item 50. The kit according to any one of the preceding items, characterized in that the supporting member (202) is made of a plastic material, such as a plastic material, wherein the thickness of the plastic material is less than about 2 mm, such as 1 mm or less.

Item 51. The kit according to any one of the preceding items, characterized in that said supporting member is configured with one proximal end (203) configured as a finger grip and opposite distal end (204) to which said sample collection pad (201) is attached.

Item 52. The kit according to any one of the preceding items, characterized in that the sample collection pad (201, 101) is made of a cellulose material, a cellulose derivative such as nitrocellulose, polyether sulfone, polyethylene, nylon polyvinylidene fluoride (PVDF), polyester, polypropylene, glass fibers, cotton, or cloth and optionally that the sample collection pad (201, 101), wherein said collection pad is optionally pre-treated with a blocking buffer.

Item 53. The kit according to any one of the preceding items, characterized in that the sample collection pad (201, 101) is pre-treated with a blocking buffer, such as a PBS buffer comprising 1% BSA or a buffer comprising 10 mM Borate, 3% BSA, 1% PVP-40 and 0.25% Triton X100 pH 8.0

Item 54. The kit according to any one of the preceding items, characterized in that the sample collection pad (201, 101) is in the form a layer of one or more sheets or the like, such as a layer of two sheets.

Item 55. The kit according to any one of the preceding items, characterized in that the cassette (300) comprises a port (318) configured to accept the distal end of said swab such that the position of the sample collection pad (201, 101, 301) in the cassette (300) is secured.

Item 56. The kit according to any one of the preceding items, characterized in that the cassette (300) is constructed so as to form a porous support assembly (100, 301), when it is mated with the sample collection pad attached to said swab, wherein the cassette (300) mated with the sample collection pad comprise an elution zone (101), a conjugate zone such as in the form of a conjugate pad (102) and a detection area (DA) and optionally a wicking pad (104), wherein the detection area (DA) comprise a detection zone (105) containing one or more affinity molecule(s) for selectively retaining one or more test analyte(s) and optionally an indicator zone (106) containing one or more affinity molecule(s) for selectively retaining one or more indicator affinity molecule(s) and optionally a inspection window (317) configured for visual inspection of the detection area (DA).

Item 57. Method for detecting the presence or quantity of one or more test analytes, the method comprising the following steps:
a) provide a sample collection pad (201) as defined in any of the preceding items, such as separate swab (200) comprising said sample collection pad (201), wherein said sample pad comprises a test sample obtained from the a skin surface of a mammal such as a human being using said separate swab;
b) insert said sample collection pad containing said test sample in the cassette (300) comprising a porous support assembly (100, 301) and blister pack (302) comprising a buffer solution and a housing (306) covering said blister as defined in any of the preceding items,
c) actuating the housing (306) to pierce and dispense said buffer solution, and
d) allowing buffer solution and test sample to migrate through the porous support assembly (100, 301).

Item 58. The method according to item 57, wherein the sample collection pad (201, 101) is pre-treated with a blocking buffer, such as a PBS buffer comprising 1% BSA or a buffer comprising 10 mM Borate, 3% BSA, 1% PVP-40 and 0.25% Triton X100 pH 8.0.

Item 59. The method according to item 57 or 58, wherein the sample collection pad (201, 101) is prewetted prior to sample collection.

Item 60. The method according to any one of items 57 to 59, wherein said test analytes are IL-8, IL-1a and IL-1 RA.

Item 61. The method according to any one of items 57 to 60 further comprising a step e) of capturing an image of the detection area (DA) and transmitting said image to a computer system comprising an image processor and a database, wherein the image features are extracted from the image by the image processor and said image features is stored in said database and, wherein said computer system generates a least one output datum based on said image features, where the image is captured using a mobile device, such a cell phone configured to capture images and output datum generated by the computer system is transferred to said mobile device.

Item 62. The method according to any one of items 57 to 61, comprising the step of determining the presence of one or more test analytes by visual inspection of the detection area (DA).

Item 61. A method for evaluating a skin condition of a subject using an electronic device comprising a camera, said method comprising the steps of:
(i) obtaining via a user interface, a request for evaluation of the skin condition of a subject,
(ii) obtaining via the user interface, user input comprising:
   data identifying said subject,
   at least one image of the detection area (DA) of the detection area (DA) of the lateral flow device according to any one of items 1 to 56 used for detecting one or more test analytes, obtained from the surface of the skin, said at least one image being recorded using the camera arranged at a distance from the test device;
(iii) registering said subject based on the data identifying the subject,
(iv) processing the user input including said at least one image and
(v) generating an evaluation of the skin condition of a subject;
(vi) outputting said evaluation of the skin condition and/or outputting a recommendation for skin care corresponding to the skin condition to the user interface.

### EXAMPLES

### Example 1

The effect the blister pack volume on the assay performance was tested.

The cassettes parts, a test strip and blisters with PBS were assembled to from a flow device.

Blister packs filled with different volumes were tested to assess the effects of buffer volume of the test performance. Blister packs with different buffer volumes a were inserted into cassette and running buffer was released to a strip in the cassette. Volumes starting 145 μl and up showed complete test run, smaller blister fill volumes were not enough for the test to run properly (FIGS. 9 and 10).

The data demonstrate that 105 μl fill volume blisters were not sufficient to even start the test run and 125 μl blisters allowed only partial test run. When tested without the swab, the 145 μl blister fill volume was adequate for the test to run properly. When tested with a swab, 135 μl fill volume blisters dispensed adequate amount of PBS (running buffer) to allow complete test strip run.

### Example 2

Cassette parts, test strips and blisters with 145 ul of PBS were assembled to form a series of lateral flow devices. Subsequently, those lateral flow devices were used to assess the functionality of different swabs, consisting of different thickness of the plastic handle (or carrier), and/or containing sampling pads with different materials.

Test functionality was assed as signal intensities on the test strip after 30 minutes, as analyzed with a Qiagen ESEQuant LR3

Tests were performed using 1) standard protein solutions and 2) samples from skin.

TABLE 1
Standard protein solutions used to test the swabs. 30 μl of
standard solutions were applied to each sample pad.

| Standard | Standard protein concentration (ng/ml) | | |
|---|---|---|---|
| solution | IL8 | IL1A | IL1RA |
| St1 | 10 ng/ml | 100 ng/ml | 200 ng/ml |
| St2 | 1 ng/ml | 10 ng/ml | 20 ng/ml |
| Neg | 0 ng/ml | 0 ng/ml | 0 ng/ml |

30 μl of Standard solutions were pipetted onto each different sample pad (2 parallels for each sample). In addition, 40 μl diluent was added to each sample pad to allow sufficient wetting of the sample pad. Subsequently, sample pads were inserted into test cassettes containing 4 mm test strips and the buttons (and housings of the blister) were pressed to release the running buffer from the 145 μl fill volume blisters in the cassette tops. Signal intensities were read after 30 minutes with Qiagen ESEQuant LR3.

For skin analysis 30 μl of running buffer was pipetted onto a sample pad and Sample pads were swabbed on skin (cheek of 1 volunteer, in 2 or 1 parallels each) for about 5 seconds. The sample pad with the carrier was inserted into a cassette containing a 4 mm test strip. To start the test, the button was pressed to release the running buffer from a blister pack filled with 145 μl buffer in the cassette top. Signal intensities were read after 30 minutes with Qiagen ESEQuant LR3.

Swabs tested:
C083 swab, blocked
Sterlitech CFP 230 swab, not blocked, heat staked
Original swab handle (thin plastic, 0.02″)
0.04″ swab handle
0.06″ swab handle

TABLE 2
Results of Sterlitech CFP 230 and CO83 swabs in lateral flow testing,
running buffer (PBS) was dispensed from the blister with 145 μl fill
volume. Par 1 and Par 2 are two parallel measurements of similar
samples in two different tests.

| | | IL8 | | IL1A | | IL1RA | |
|---|---|---|---|---|---|---|---|
| | | Par1 | Par2 | Par1 | Par2 | Par1 | Par2 |
| C083 | St1 | 817.6 | 794.5 | 843.7 | 766.4 | 558 | 476.1 |
| | St2 | 474.6 | 507.4 | 547.2 | 448.3 | 172.5 | 164.7 |
| | St3 | 52.7 | failed | 386.9 | failed | 20 | failed |
| CFP230, thin | St1 | 822.1 | failed | 756.3 | failed | 507.6 | failed |
| carrier | St2 | 382 | failed | 456.1 | failed | 229 | failed |
| | St3 | failed | failed | Invalid | failed | failed | failed |
| CFP230, | St1 | 771.7 | 692.2 | 762.5 | 705 | 481.8 | 490.2 |
| 0.04″ carrier | St2 | 135.7 | 210.2 | 277.3 | 215 | 123.5 | 99.5 |
| | St3 | 0 | 0 | 233.2 | 80.7 | 24.2 | 0 |
| CFP230, | St1 | 734.2 | 813.4 | 696 | 754.2 | 360.5 | 492.8 |
| 0.06″ carrier | St2 | 407.6 | 381.5 | 358.1 | 302.8 | 114.6 | 125.2 |
| | St3 | 0 | 0 | 237.7 | 144.7 | 0 | 0 |
| C083 | Cheek | 97.5 | failed | 515.3 | Invalid | 85.5 | failed |
| | V1 | | | | | | |
| CFP230, thin | Cheek | failed | failed | failed | failed | failed | failed |
| carrier | V1 | | | | | | |
| CFP230, | Cheek | 208 | — | 210.4 | — | 158.7 | — |
| 0.04″ carrier | V1 | | | | | | |
| CFP230, | Cheek | failed | — | failed | — | failed | — |
| 0.06″ carrier | V1 | | | | | | |

The data demonstrate that many tests failed to run properly. Better runs were obtained when using thicker swab carriers, but even then not all tests run correctly. The runs are shown in FIGS. 11 to 15.

The data demonstrate:
Many tests failed to run when different sample pads (or sample pad carriers) were tested with a standard protein solution or on skin and 145 μl fill volume blisters for running buffer release. Total 9 tests out of 30 did not run properly or at all.
Running was better when thicker swab carrier was used, but even then not all tests run correctly.

### Example 3

Example 1 demonstrates that 135 μl blister fill volume of running buffer (PBS) is sufficient for the test to run, but tests with 145 μl fill volume blisters showed that many tests failed to run properly. Therefore, blister packs with larger volumes of PBS were tested and test cassettes observed visually to determine the probable reason for test malfunctioning.

Therefore. cassette parts, test strips and blisters with different volumes of PBS were assembled to form a series of lateral flow devices. Subsequently, those lateral flow devices were tested to assess the correlation between buffer volume and test functionality.

Test functionality was assed as time for the test to start running after pressing the cassette button. 30 μl of diluent was pipetted onto sample pad, sample pad was inserted into the cassette containing 4 mm test strip and button was pressed to release the running buffer from the 155 μl or 165 μl fill volume blisters in the cassette top.

Results were evaluated visually.

Swabs tested:
Sterlitech CFP 230 swab, not blocked, heat staked
Original swab handle (thin plastic, 0.02″(?))
0.04″ swab handle
0.06″ swab handle

The result of the test run are shown in FIGS. 16 to 21.

For the 155 μl blister: 4 cassettes out of 35 did not run (3 did not run at all, 1 started to run 10 minutes after piercing the blister).

For the 165 μl blister: 1 cassette out of 33 was not running at all, for few cassettes the start of the run were delayed (0.5-2 minutes) after pressing the button to release the running buffer, but were the runs completed properly.

Results in summary
Previously it was shown that 135 μl blister volume is sufficient to run the test. 145 μl blister volume resulted in many of the tests not running properly—total 9 tests out of 30 did not run properly or at all.
Increasing the blister fill volume to 155 μl or 165 μl improves the amount of tests run properly (number of failed tests 4 out of 35 or 1 out of 33, respectively).
Increasing the blister fill volume potentially flood the cassette.
It was clearly visible that droplets of the running buffer were inside the cassettes and even splashed out of the cassette.
When the buffer splashes from the blister the volume reaching the test strip and functioning properly as the running buffer differs considerably and cannot be predicted. Variations in running buffer amount will influence the test results.

Conclusion: Increasing the blister pack fill volume decreased the number of test cassettes not being able to run. However, droplets of the running buffer were observed inside the cassettes, as well as splashes of buffer outside of the cassette (data not shown). Thus a too large volume of running buffer in the blister pack can potentially flood the cassette. The data demonstrate that the variations in running buffer actually reaching the test strip actually influence the test results. Thus it seems that the volume of the buffer in the blister pack influence the functionality of the test in two ways. A too little volume affects the test for instance in incomplete running of the test. A too large volume may influence the test by introducing variability in the buffer volume that is actually absorbed by the test strip. Thus the conclusion is that the test performance critically depends on the volume of buffer in the blister pack.

### Example 4

Cassette parts, test strips and blisters with different volumes of PBS were assembled to form a series of lateral flow devices. Subsequently, those lateral flow devices were tested to assess the correlation between position of the blister pack, and fixing of the blister pack, and test functionality.

Test functionality was assed as 1) time for the test to start running, and 2) visual signs of buffer splashed into the inside of the cassette after pressing the cassette button.

Blisters with 145 μl fill volume were inserted into cassette. The cassettes were assembled in the following way:
Normal (blister aligned correctly to the center)
Misaligned (blister aligned not to the center)
Adhesive (blister aligned correctly to the center and secured with double side adhesive) The cassette were numbered. The study was blinded and the alignment was revealed after the study had been conducted.

30 μl of diluent was pipetted onto sample pad (0.04″ thick handle), sample pad was inserted into the cassette containing 4 mm test strip and button was pressed to release the running buffer from the 145 μl fill volume blisters in the cassette. The time from pressing the button to seeing visible fluid front in the cassette window was measured and recorded.

TABLE 3
Results of running cassettes with differently aligned blisters in
the cassette top. The time (seconds) from pressing the button to
seeing visible fluid front in the cassette window was measured. Fluid
visible on cassette top or bottom (when opened) is shown as “+”

| | Time from | Fluid | Fluid | | |
|---|---|---|---|---|---|
| Cas- | pressing the | drops on | drops on | | |
| sette | button to seeing | cassette | cassette | | |
| # | fluid front (sec) | top | bottom | Comments | Assembly |
| 1 | 17 | | | | adhesive |
| 2 | 36 | | | | adhesive |
| 3 | 10 | | | | misaligned |
| 4 | 7 | + | | | normal |
| 5 | 46 | | | | misaligned |
| 6 | 7 | | | | adhesive |
| 7 | 41 | + | + | | normal |
| 8 | 7 | | | | normal |
| 9 | 44 | | | | adhesive |
| 10 | 80 | + | | | misaligned |
| 11 | 19 | | | | misaligned |
| 12 | 76 | | | | normal |
| 13 | 18 | + | | | adhesive |
| 14 | 6 | + | | | adhesive |
| 15 | 10 | + | | | normal |
| 16 | 10 | | | | misaligned |
| 17 | >120 | ++ | ++ | Pink strip | normal |
| 18 | 24 | + | | | adhesive |
| 19 | 15 | | | | normal |
| 20 | 16 | | | | adhesive |
| 21 | 15 | | | | misaligned |
| 22 | 31 | | | | misaligned |
| 23 | 8 | + | | | adhesive |
| 24 | 7 | + | | | misaligned |
| 25 | 15 | | | | normal |
| 26 | 13 | + | | | misaligned |
| 27 | 13 | | | | adhesive |
| 28 | 10 | | | | misaligned |
| 29 | 60 | | | | misaligned |
| 30 | 15 | | | | adhesive |
| 31 | 40 | | | | adhesive |
| 32 | 14 | | | | normal |
| 33 | 5 | | | | misaligned |
| 34 | 25 | | | | normal |
| 35 | 10 | + | | | misaligned |
| 36 | 28 | | + | | adhesive |
| 37 | 20 | + | | | misaligned |
| 38 | 18 | | | | misaligned |
| 39 | 19 | + | + | | adhesive |
| 40 | 25 | | | | normal |
| 41 | 12 | + | | | normal |
| 42 | 19 | + | | | normal |
| 43 | 7 | + | | | normal |
| 44 | 11 | | | | adhesive |
| 45 | 7 | + | | | misaligned |
| 46 | 7 | + | | | normal |
| 47 | 8 | + | | | adhesive |
| 48 | 36 | | | | misaligned |
| 49 | 16 | + | | | misaligned |
| 50 | 17 | | | | adhesive |
| 51 | 5 | | | | normal |
| 52 | 14 | | | | normal |
| 53 | 19 | | | | adhesive |
| 54 | 42 | | | | normal |
| 55 | 19 | | | | misaligned |
| 56 | 8 | + | | | normal |
| 57 | 12 | | | | adhesive |
| 58 | 6 | + | + | | normal |
| 59 | 8 | + | | | adhesive |
| 60 | 27 | | | | misaligned |

TABLE 4
Summary of the results in Table 3

| | Adhesive | Misaligned | Correctly aligned |
|---|---|---|---|
| Average run time (sec) | 18.3 | 22.95 | 18.68 |
| No of tests ≤15 sec | 9 | 9 | 13 |
| No of tests ≤30 sec | 17 | 15 | 16 |
| No of tests ≥1 min | 0 | 2 | 2 |

Four cassettes started running 1 minute or more after pressing the button, 1 of them more than 2 minutes later (the run as not completed). Two of the cassettes with ≥1 minute start were correctly aligned and 2 were misaligned, none were with adhesive.

Tests starting 1 minute after pressing the button were considered as not acceptable.

In the current experiment the blisters secured with adhesive in the correct position worked the best: 17/20 tests start time≤30 sec and no tests with over 1 minute. The data confirms the importance of proper alignment of the blister.

### Example 5

Cassette parts, test strips and blisters with 145 μl of PBS were assembled to form a series of lateral flow devices. Subsequently, those lateral flow devices were tested to assess the correlation between position of the blister pack, composition of the cassette, fixing of the blister pack, and test functionality.

Test functionality was assed as time for the test to start running.

The cassettes were assembled in the following way:
Adhesive (blister aligned correctly to the center and secured with double side adhesive, small round hole in the middle of the adhesive)
Blisters with larger edge (so that the blister can not move in the cassette and be misalign)
Hydrophobic coated cassettes, normal blisters.

30 μl of diluent was pipetted onto a sample pad (0.04″ thick handle). Subsequently, the sample pad was swabbed briefly on skin and inserted into the cassette containing a 4 mm test strip. Then, the button was pressed to release the running buffer from the 145 μl fill volume blisters in the cassette top. The time from pressing the button to seeing visible fluid front in the cassette window was measured and recorded.

TABLE 5
Results of running cassettes with differently aligned blisters
in the cassette top. The time from pressing the button to seeing
visible fluid front in the cassette window was measured.

| | Time from pressing the button |
|---|---|
| Assembly type | to seeing fluid front (sec) |
| adhesive | 16 |
| adhesive | 6 |
| adhesive | 12 |
| adhesive | 7 |
| adhesive | 7 |
| adhesive | 3 |
| adhesive | 11 |
| adhesive | 51 |
| adhesive | 6 |
| adhesive | 9 |
| adhesive | 13 |
| adhesive | 27 |
| adhesive | 6 |
| adhesive | 27 |
| adhesive | 4 |
| adhesive | 5 |
| adhesive | 4 |
| adhesive | 12 |
| adhesive | 7 |
| adhesive | 9 |
| larger diameter blister | 15 |
| larger diameter blister | — |
| larger diameter blister | 20 |
| larger diameter blister | 15 |
| larger diameter blister | 13 |
| larger diameter blister | 9 |
| larger diameter blister | >120 |
| larger diameter blister | 7 |
| larger diameter blister | 7 |
| larger diameter blister | — |
| Hydrophobic cassette | 36 |
| Hydrophobic cassette | 21 |
| Hydrophobic cassette | 15 |
| Hydrophobic cassette | 18 |
| Hydrophobic cassette | 30 |
| Hydrophobic cassette | 29 |
| Hydrophobic cassette | 27 |
| Hydrophobic cassette | 50 |
| Hydrophobic cassette | 49 |
| Hydrophobic cassette | 21 |

TABLE 6
Summary of the results in Table 5 (Current testing) and from a
similar testing experiment using different parameters for assembly of
the blister pack into the cassette. All results are with 0.04″ swab handle
and 145 μl blisters. Tests starting over 2 minute after pressing the
button or not starting at all were used as 120 second in calculating the
average start time.

| | Previous testing | | | Current testing | | |
|---|---|---|---|---|---|---|
| | | | Blister | Blister | | |
| | | | w/ | w/ | | |
| | | Mis- | adhesive | adhesive | Wide | Hydro- |
| Assembly | Normal | aligned | (square | (round | edge | phobic |
| type | blister | blister | hole) | hole) | blister * | cassette ** |
| No of | 20 | 20 | 20 | 20 | 10 | 10 |
| cassettes | | | | | | |
| run | | | | | | |
| Average | 23.75 | 22.95 | 18.3 | 12.1 | 44.6 | 29.6 |
| test run | | | | | | |
| start time | | | | | | |
| (sec) | | | | | | |
| No of tests | 13 | 9 | 9 | 16 | 6 | 1 |
| ≤15 sec | | | | | | |
| (0-15) | | | | | | |
| No of tests | 16 | 15 | 17 | 19 | 7 | 7 |
| ≤30 sec | | | | | | |
| (0-30) | | | | | | |
| No of tests | 2 | 3 | 3 | 1 | 0 | 3 |
| 31-59 sec | | | | | | |
| No of tests | 2 | 2 | 0 | 0 | 3 | 0 |
| ≥1 min | | | | | | |
| Total tests | 13/20 = | 9/20 = | 9/20 = | 16/20 = | 6/10 = | 1/10 = |
| very good | 65% | 45% | 45% | 80% | 60% | 10% |
| (0-15 sec) | | | | | | |
| Total tests | 16/20 = | 15/20 = | 17/20 = | 19/20 = | 7/10 = | 7/10 = |
| good (0- | 80% | 75% | 85% | 95% | 70% | 70% |
| 30 sec) | | | | | | |
| Total tests | 18/20 = | 18/20 = | 20/20 = | 20/20 = | 7/10 = | 10/10 = |
| OK (0- | 90% | 90% | 100% | 100% | 70% | 100% |
| 59 sec) | | | | | | |

* Very hard to press the button, edges of blister not even and hinder assembly assembly and pressing
** Still wet under blister. Very hard to assemble the cassette. Cassette smells of the hydrophobic coating.

TABLE 7
Results of running cassettes with different blisters in the
cassette top. The time from pressing the button to seeing
visible fluid front in the cassette window was measured.

| | Time from pressing | | |
|---|---|---|---|
| | the button to seeing | | |
| Assembly type | fluid front (sec) | | |
| Blister with | 11 | Average run time (sec) | 21.16667 |
| PBS | 24 | No of tests ≤15 sec | 2 |
| | 16 | No of tests ≤30 sec | 5 |
| | 34 | No of tests 31-59 sec | 1 |
| | 13 | No of tests ≥1 min | 0 |
| | 29 | | |
| Blister with | 28 | Average run time (sec) | 34.5 |
| Senova diluent | 31 | No of tests ≤15 sec | 0 |
| | 32 | No of tests ≤30 sec | 3 |
| | 24 | No of tests 31-59 sec | 4 |
| | 67 | No of tests ≥1 min | 1 |
| | 35 | | |
| | 36 | | |
| | 23 | | |
| Blister with | 15 | Average run time (sec) | 13.2 |
| PBS + | 6 | No of tests ≤15 sec | 8 |
| adhesive | 8 | No of tests ≤30 sec | 9 |
| | 12 | No of tests 31-59 sec | 1 |
| | 32 | No of tests ≥1 min | 0 |
| | 18 | | |
| | 7 | | |
| | 15 | | |
| | 7 | | |
| | 12 | | |
| Blister with | 17 | Average run time (sec) | 25.2 |
| Senova | 24 | No of tests ≤15 sec | 1 |
| diluent + | 70 | No of tests ≤30 sec | 9 |
| adhesive | 25 | No of tests 31-59 sec | 0 |
| | 27 | No of tests ≥1 min | 1 |
| | 17 | | |
| | 24 | | |
| | 17 | | |
| | 14 | | |
| | 17 | | |

The experiments reveal that 90% of tests run in cassettes with blisters assembled into the cassette without using adhesive, whereas 10% of tests did not run or started minute after breaking the blister.

Similar results were obtained when blister packs have either been deliberately aligned or misaligned, indicating that either the exact blister position is not the critical parameter or that the blisters can move inside the seat of the cassette during the test procedure.

Blisters adhered with adhesive (square round in the middle) had 100% cassettes running within 1 minute, 85% starting within first 30 second but only 45% starting within first 15 seconds. The average run start time is shorter when blisters were adhered with adhesive (18.3 seconds compared to 23-24 seconds when not adhered).

Testing with wider edge blisters had only 70% cassettes running within 1 minute (2 tests out of 10 did not run at all), which indicates that a wider edge of the blister pack hinders, not aids, in test running.

Hydrophobic coating of the test cassette does lead to 100% cassettes running in 1 minute, but only 70% started in the first 30 seconds and 10% in the first 15 seconds leading to high average run start time.

Adhesive with a small round hole in the middle resulted in 100% of the cassettes running, with 95% starting within 30 seconds and 80% starting within the first 15 seconds. The average running start time is also considerably shorter; 12.1 seconds for round hole adhesive compared to 18.3 seconds with adhesive with a larger square hole, and 23-24 seconds when not adhered.

### Example 6

Cassette parts, test strips and blisters with 145 μl of PBS were assembled to form a series of lateral flow devices. Subsequently, those lateral flow devices were tested for functionality to assess the correlation between the functionality of single sided swabs versus double sided swabs. In addition, the fixing of the blister pack was tested in this experimental setting. Test functionality was assed as time for the test to start running.

Second layer of CFP230 sample collection pad was adhered to the other side of a single-sided swab using PVA glue. 50 μl of diluent was pipetted onto sample pad (0.04″ thick handle), sample pad was swabbed briefly on skin and inserted into the cassette containing 4 mm test strip and button was pressed to release the running buffer from the 14 μl fill volume blisters in the cassette top.

TABLE 8

| | | 1-sided swab | 2-sided swab |
|---|---|---|---|
| Blister w/PBS, no | Start time average | 23.75 | 21.2 |
| adhesive | % started in 15 sec | 65% | 33% |
| | % started in 30 sec | 80% | 83% |
| | % started in 60 sec | 90% | 100% |
| Blister w/PBS, + | Start time average | 12.1 | 13.2 |
| adhesive | % started in 15 sec | 80% | 80% |
| | % started in 30 sec | 95% | 90% |
| | % started in 60 sec | 100% | 100% |

Using double sided swabs does not improve test running properties compared to regular single sided swabs. Using adhesive to adhere blisters still improves test running.

### Example 7

Cassette parts, test strips and blisters with PBS or Senova buffer were assembled to form a series of lateral flow devices. In between the cassette top-plate and the blister pack, a shim was introduced with different diameters in opening. Subsequently, those lateral flow devices were tested to assess the correlation between shim opening size and test functionality.

Test functionality was assessed as 1) force top press the housing down and actuate the piercing of the blister, and 2) time for the test to start running, and 3) differences between running buffers.

TABLE 9
Force applied

| | 2 mm | 3 mm | 4 mm | 5 mm | Control |
|---|---|---|---|---|---|
| Test # | ID (N) | ID (N) | ID (N) | ID (N) | (N) |
| 1 | 44.50 | 29.82 | 34.49 | 38.94 | 26.70 |
| 2 | 50.06 | 31.15 | 35.60 | 34.49 | 24.48 |
| 3 | 33.38 | 28.48 | 39.83 | 35.60 | 30.48 |
| 4 | 31.15 | 29.82 | 43.39 | 44.50 | 35.60 |
| 5 | 38.72 | 31.15 | 36.27 | 41.61 | 24.03 |
| avg | 39.56 | 30.08 | 37.91 | 39.03 | 28.26 |
| std | 6.99 | 1.00 | 3.27 | 3.71 | 4.32 |

TABLE 10
Time for run to begin (seconds)

| Test | 2 mm ID (sec) | 3 mm ID (sec) | 4 mm ID (sec) | 5 mm ID (sec) |
|---|---|---|---|---|
| 1 | 7 | 5 | 7 | 8 |
| 2 | 5 | 3 | 4 | 6 |
| 3 | 5 | 4 | 9 | 6 |
| 4 | 12 | 7 | 5 | 6 |
| 5 | 7 | 8 | 7 | 7 |
| Avg | 7.20 | 5.40 | 6.40 | 6.60 |
| Std | 2.86 | 2.07 | 1.95 | 0.89 |

The data demonstrate that a shim with a central hole having a diameter of 3 mm is optimal.

TABLE 11
Comparing PBS buffer and Senova buffer

| Test | PBS 3 mm ID | Senova 3 mm ID |
|---|---|---|
| 1 | 29.82 | 36.71 |
| 2 | 31.15 | 34.49 |
| 3 | 28.48 | 42.28 |
| 4 | 29.82 | 42.28 |
| 5 | 31.15 | 45.61 |
| avg | 30.08 | 40.27 |
| std | 1.00 | 4.07 |

The data suggests that Senova buffer increases the force necessary to actuate the piecing of the blister to release the buffer. The data also suggests that Senova buffer delays the run.

1. A kit for detecting the presence or quantity of one or more test analytes within a test sample obtained from a skin surface of a mammal, the kit comprising:
a) a lateral flow assay device comprising a cassette comprising one or more porous elements forming a porous support assembly, wherein said cassette is configured to receive and hold a sample collection pad, wherein said sample collection pad is configured to be in contact with said porous support assembly when said sample pad is inserted in said cassette,
b) a blister pack, wherein said blister contains a buffer solution, wherein said cassette is configured to receive said blister pack, and
c) a sample collection pad configured to be used for collecting said test sample.

2-20. (canceled)

21. The kit according to claim 1, wherein said cassette comprises an upper part and a lower part, wherein said upper part of said cassette comprises a seat configured to receive said blister pack, and wherein said seat is optionally having a diameter equal to or approximately equal to the diameter of the lower part of the blister pack.

22. The kit according to claim 1, wherein the upper part of said cassette comprises a seat configured to receive said blister pack and a housing covering said blister, wherein said housing optionally comprises alignment members configured to align and position the blister pack in the seat.

23. The kit according to claim 1, wherein the housing comprises an upper part an a lower part, wherein the housing comprises a least one first locking member and a least one second locking member protruding from the lower part of the housing, and wherein said at least one first locking member is optionally longer than said at least one second locking member.

24. The kit according to claim 1, wherein said at least one first locking member secures the housing in the cassette in a position covering said blister pack and said at least one second locking member secures the housing in the cassette in the actuated position.

25. The kit according to claim 1, wherein the upper part of said cassette comprises a seat configured to receive said blister pack and a shim, and wherein said shim is positioned between the blister pack and the bottom of said seat.

26. The kit according to claim 1, wherein said shim is adhesive on the side facing down towards the lower part of the cassette, on the side face up towards the blister pack or both sides.

27. The kit according to claim 1, wherein said shim has a central hole having a diameter in the range of 2 mm to 5 mm.

28. The kit according to claim 1, wherein said shim is an integrated part of the seat.

29. The kit according to claim 1, wherein the bottom of the seat comprises at least one protruding part, wherein said at least one protruding part is configured to pierce the bottom part of the blister pack and release the buffer solution from the blister pack when said housing is actuated by pressing the housing towards the seat and, wherein said protruding part(s) optionally comprises a central tubular duct extending from the top of the protruding part to and through the bottom of the seat.

30. The kit according to claim 29 wherein said protruding part(s) comprising a central tubular duct is configured to allow passage of buffer solution from the blister pack when said housing is actuated.

31. The kit according to claim 1, wherein said blister pack comprises a blister dome and a blister bottom film, and wherein said blister bottom film comprises an adhesive on the outer surface.

32. The kit according to claim 1, wherein the volume of the buffer solution in said blister pack is at least 130 microlitres.

33. The kit according to claim 1, wherein said seat comprises an aperture configured to allow the buffer solution released from the blister pack to come in contact with the porous support assembly.

34. The kit according to claim 1, wherein said porous support assembly comprises a detection area and said cassette comprises an inspection window configured for visual inspection of the detection area, wherein said upper surface of the upper part of the cassette optionally comprises at least one reference point suitable for image detection of the detection area and/or image detection of the orientation of the detection area.

35. The kit according to claim 1, wherein said cassette comprises a tablet, capsule or sachet of desiccant.

36. The kit according to any claim 1, wherein said cassette comprises a sample port configured to receive said sample collection pad, said sample collection pad optionally attached to a supporting member, wherein said sample port optionally comprises one or more rails configured position the sample collection pad over and in contact with the said porous support assembly.

37. The kit according to claim 36, wherein said sample collection pad, or sample collection pad attached to the supporting member, has a thickness that prevents buffer solution from leaking out through the sample port when the sample collection pad or sample collection pad attached to the supporting member is inserted in the sample port.

38. A method for detecting the presence or quantity of one or more test analytes, the method comprising:
a) providing a sample collection pad as defined in claim 1, wherein said sample pad comprises a test sample obtained from the a skin surface of a mammal;
b) inserting said sample collection pad containing said test sample in the cassette comprising a porous support assembly and blister pack comprising a buffer solution and a housing covering said blister as defined in claim 1,
c) actuating the housing to pierce and dispense said buffer solution, and
d) allowing buffer solution and test sample to migrate through the porous support assembly and, optionally:
e) capturing an image of the detection area and transmitting said image to a computer system comprising an image processor and a database, wherein the image features are extracted from the image by the image processor and said image features are stored in said database and, wherein said computer system generates a least one output datum based on said image features, where the image is captured using a mobile device, and output datum generated by the computer system is transferred to said mobile device.

39. A method for evaluating a skin condition of a subject using an electronic device comprising a camera, said method comprising:
(vii) obtaining via a user interface, a request for evaluation of the skin condition of a subject,
(viii) obtaining via the user interface, user input comprising:
data identifying said subject,
at least one image of the detection area of the lateral flow device according to claim 1 used for detecting one or more test analytes, obtained from the surface of the skin, said at least one image being recorded using the camera arranged at a distance from the test device;
(ix) registering said subject based on the data identifying the subject;
(x) processing the user input including said at least one image;
(xi) generating an evaluation of the skin condition of a subject; and
(xii) outputting said evaluation of the skin condition and/or outputting a recommendation for skin care corresponding to the skin condition to the user interface.
